(19) United States
(12) Patent Application Publication
(10) Pub. No.: US 2021/0252192 A1
(43) Pub. Date: Aug. 19, 2021

(54) USE OF A HYALURONIC ACID-BASED HYDROGEL FOR TREATMENT OF VOLUMETRIC MUSCLE LOSS INJURY

(71) Applicant: University of Virginia Patent Foundation, Charlottesville, VA (US)

(72) Inventors: George Joseph Christ, Crozet, VA (US); Kevin Healy, Berkeley, CA (US); Juliana Amaral Passipieri, Charlottesville, VA (US); Shane Andrew Browne, Berkeley, CA (US)

(21) Appl. No.: 17/049,237

(22) PCT Filed: Apr. 22, 2019
(86) PCT No.: PCT/US2019/028558

§ 371 Date: Oct. 20, 2020

Related U.S. Application Data
(60) Provisional application No. 62/660,379, filed on Apr. 20, 2018.

(51) Int. Cl. A61L 27/52; A61L 27/26; A61L 27/54; A61K 47/69; A61P 21/00; A61K 47/65

(52) U.S. Cl. CPC A61L 27/52; A61L 27/26; A61L 27/54; A61L 2430/30; A61P 21/00; A61K 47/65; A61K 47/6903

(57) ABSTRACT

A method of treating volumetric muscle loss (VML) injury involving the administration of a hydrogel that is free of biological cells and growth factors is described. The hydrogel can comprise a crosslinked hydrogel matrix further comprising heparin wherein the hydrogel can be administered to a subject in precursor form and allowed to crosslink in situ in a muscle injury site or administered in a lyophilized powder, sheet or disc form. The presently disclosed subject matter further describes lyophilized hydrogel matrix materials, such as sheets, wafers and powders, that can be used to treat VML injury.

### CROSS-REFERENCE TO RELATED APPLICATIONS

This application claims benefit of U.S. Provisional Patent Application Ser. No. 62/660,379, filed Apr. 20, 2018, herein incorporated by reference in its entirety.

### TECHNICAL FIELD

The subject matter disclosed herein relates generally to methods of treating volumetric muscle loss (VML) injury and to regenerative therapeutic compositions for muscle regeneration. More particularly, the subject matter disclosed herein relates to hyaluronic acid-based hydrogel materials, such as hydrated or freeze-dried suturable gels, sheets, and powders, and their use in repairing, regenerating and/or remodeling skeletal muscle to treat trauma and diseases or conditions resulting in loss of skeletal muscle and/or skeletal muscle function.

### BACKGROUND

Volumetric muscle loss (VML) is the traumatic or surgical loss of skeletal muscle that results in irrecoverable structural and functional impairment, ranging from disfigurement to life-long disability. Patients with VML cannot recover because their bodies cannot regenerate the lost muscle. Annually, millions of automobile accidents result in traumatic injury to the extremities. In addition, a majority of battlefield injuries are musculoskeletal in nature. Congenital VML also plays a role. Each year, 4,440 babies are born with cleft lip with or without cleft palate and 2,650 babies are born with cleft palate.

Current treatment options include functional free muscle transfer to the injury site and physical therapy. However, the results of free muscle transfer are inconsistent and can depend on the skill of the surgeon. Physical therapy has not been shown to significantly improve recovery after VML and does not restore skeletal muscle fibers. While there are some tissue engineering (TE) approaches currently under development to treat VML, there is significant room for therapeutic improvement in the timeliness and magnitude of functional recovery. For example, there remains a need for additional methods and approaches to accelerate cellular organization and muscle tissue formation, vascularization, and function for in vivo therapeutic treatment of VML.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a method of treating volumetric muscle loss (VML) injury, the method comprising: (a) providing a subject in need of VML treatment; and (b) administering to the subject a regenerative hydrogel system, wherein said regenerative hydrogel system comprises a growth factor recruitment moiety, optionally heparin or a derivative or copolymer thereof, and wherein the regenerative hydrogel system is free of exogenous growth factors and biological cells, whereby the VML in the subject is treated, optionally wherein the treating provides between about 20 percent (%) and about 99% muscle function recovery, further optionally wherein the treating provides between about 50% and about 99% muscle function recovery.

In some embodiments, the regenerative hydrogel system comprises a plurality of hydrogel matrix precursors, wherein said precursors comprise: (i) one or more hydrogel polymers; (ii) a growth factor recruitment moiety, optionally heparin or a derivative thereof, further optionally wherein the heparin is conjugated to one or more hydrogel polymers; and (iii) a proteolytically cleavable cross-linker peptide; and wherein the administering of step (b) comprises administering the plurality of hydrogel matrix precursors to a muscle injury site in the subject, whereby the precursors form a crosslinked hydrogel matrix in situ in the muscle injury site, wherein said crosslinked hydrogel matrix comprises one or more hydrogel polymers, wherein said one or more hydrogel polymers comprises a growth factor recruitment moiety-conjugated hydrogel polymer, and wherein the proteolytically cleaveable cross-linker agent links one of the one or more hydrogel polymers to another of the one or more hydrogel polymers. In some embodiments, the administering comprises placing a removable mold in the muscle injury site prior to the administering of step (b) to define an area where the crosslinked hydrogel matrix is to be formed and where the plurality of hydrogel precursors are to be administered; and wherein the method further comprises removing the mold from the injury site following the administration of step (b), optionally wherein the mold is removed about 10 minutes after the administering of step (b). In some embodiments, the method further comprises suturing the crosslinked hydrogel matrix in place in the muscle injury site.

In some embodiments, the regenerative hydrogel system comprises a lyophilized crosslinked hydrogel matrix material comprising: (i) one or more hydrogel polymers, (ii) a growth factor recruitment moiety, optionally heparin, conjugated to one or more hydrogel polymer; and (iii) a proteolytically cleavable cross-linker peptide, wherein the proteolytically cleavable cross-linker peptide links one of the one or more hydrogel polymers to another of the one or more hydrogel polymers. In some embodiments, the lyophilized crosslinked hydrogel matrix material is administered to a muscle injury site in the subject as a sheet or disc comprising the lyophilized crosslinked hydrogel matrix material. In some embodiments, the method further comprises suturing the sheet or disc in place in the muscle injury site. In some embodiments, the lyophilized crosslinked hydrogel matrix material is administered to a muscle injury site in the subject in powder form.

In some embodiments, the one or more hydrogel polymers comprise an acrylated hyaluronic acid polymer (HyA). In some embodiments, the one or more hydrogel polymers further comprise a hydrogel polymer conjugated to a cell adhesion peptide. In some embodiments, the cell adhesion peptide comprises the amino acid sequence RGD. In some embodiments, the cell adhesion peptide comprises the amino acid sequence CGGNGEPRGDTYRAY (SEQ ID NO: 2).

In some embodiments, the proteolytically cleavable cross-linker peptide comprises the amino acid sequence CQPQGLAKC (SEQ ID NO: 1). In some embodiments, the growth factor recruitment moiety comprises heparin, optionally a high molecular weight heparin (HMWH) or a derivative or copolymer thereof, further optionally wherein the HMWH has a weight average molecular weight (MW_{w}) of between about 6 kilodaltons (kDa) and about 12 kDa.

In some embodiments, administration of the regenerative hydrogel system provides improved muscle recovery compared to a non-treated muscle injury, optionally wherein said improved muscle recovery comprises one or more of increased muscle mass compared to a non-treated muscle injury, increased muscle volume compared to a non-treated muscle injury, improved muscle vascularization compared to a non-treated injury, or improved muscle function compared to a non-treated muscle injury. In some embodiments, administration of the regenerative hydrogel system provides increased muscle mass and/or increased muscle function compared to a non-treated muscle injury within eight to twelve weeks after administration. In some embodiments, the subject is a human.

In some embodiments, the presently disclosed subject matter provides a lyophilized crosslinked hydrogel matrix comprising (i) one or more hydrogel polymers, (ii) a growth factor recruitment moiety, optionally heparin, conjugated to one or more hydrogel polymer; and (iii) a proteolytically cleavable cross-linker peptide, wherein the proteolytically cleavable cross-linker peptide links one of the one or more hydrogel polymers to another of the one or more hydrogel polymers. In some embodiments, the one or more hydrogel polymers comprises an acrylated hyaluronic acid polymer (HyA). In some embodiments, the growth factor recruitment moiety comprises heparin, optionally a high molecular weight heparin (HMWH), further optionally wherein the HMWH has a weight average molecular weight (MW_{w}) of between about 6 kilodaltons (kDa) and about 12 kDa.

In some embodiments, the one or more hydrogel polymers further comprises a cell adhesion peptide conjugated to a hydrogel polymer. In some embodiments, the cell adhesion peptide comprises the amino acid sequence RGD. In some embodiments, the cell adhesion peptide comprises the amino acid sequence CGGNGEPRGDTYRAY (SEQ ID NO: 2).

In some embodiments, the proteolytically cleavable cross-linker peptide comprises the amino acid sequence CQPQGLAKC (SEQ ID NO: 1).

In some embodiments, the presently disclosed subject matter provides a sheet or disc comprising the lyophilized crosslinked hydrogel matrix. In some embodiments, the sheet or disc has a thickness of between about 1 millimeter (mm) and about 10 mm.

In some embodiments, the presently disclosed subject matter provides a powder comprising particles of the lyophilized crosslinked hydrogel matrix, optionally wherein said powder has an average particle size of between about 50 micrometers (μm) and about 500 μm.

Accordingly, it is an object of the presently disclosed subject matter to provide methods of treating VML injury and to provide freeze-dried hydrogel compositions that can be used to treat VML injury.

These and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, drawings and examples.

### BRIEF DESCRIPTION OF THE DRAWINGS

FIG. 1A is a photographic image of the gross appearance of an exemplary healthy/uninjured rat tibialis anterior (TA) muscle.

FIG. 1B is a photographic image of the gross appearance of an exemplary rat TA muscle twelve weeks after surgery to mimic a 20 percent (%) by mass volumetric muscle loss (VML) injury.

FIG. 1C is a photographic image of the gross appearance of an exemplary rat TA muscle twelve weeks after surgery to mimic a 20% by mass VML injury and repair using a hydrogel treatment of the presently disclosed subject matter.

FIG. 1D is a representative transverse image of an exemplary healthy/uninjured rat TA muscle. Tissue was stained with hematoxylin and eosin (H & E) stain. The scale bar in the lower right of the image represents 2000 micrometers (μm).

FIG. 1E is a representative transverse image of an exemplary rat TA muscle twelve weeks after surgery to mimic a 20% by mass VML injury. Tissue was stained with H & E stain. The scale bar in the lower right of the image represents 2000 μm.

FIG. 1F is a representative transverse image of an exemplary rat TA muscle twelve weeks after surgery to mimic a 20% by mass VML injury and repair using a hydrogel treatment of the presently disclosed subject matter. Tissue was stained with H & E stain. The scale bar in the lower right of the image represents 2000 μm.

FIG. 1G is a representative transverse image of an exemplary healthy/uninjured rat TA muscle. Tissue was stained with H & E stain. The scale bar in the lower right of the image represents 100 μm.

FIG. 1H is a representative transverse image of an exemplary rat TA muscle twelve weeks after surgery to mimic a 20% by mass VML injury. Tissue was stained with H & E stain. The scale bar in the lower right of the image represents 100 μm.

FIG. 1I is a representative transverse image of an exemplary rat TA muscle twelve weeks after surgery to mimic a 20% by mass VML injury and repair using a hydrogel treatment of the presently disclosed subject matter. Tissue was stained with H & E stain. The scale bar in the lower right of the image represents 100 μm.

FIG. 1J is a graph showing the average ratio of TA weight (in milligrams (mg)) to total body weight (in grams (g)) in eleven healthy/uninjured control rats (CTRL), five rats twelve weeks after surgery to mimic a 20% by mass VML injury but not treated (NR), and six rats after surgery to mimic a 20% by mass VML injury and treatment with a hydrogel of the presently disclosed subject matter. Data are presented as the mean±standard error of the mean (SEM). ** significantly different at p<0.01, using Sidak's post-hoc after performing one-way analysis of variance (ANOVA).

FIG. 2A is a graph showing the baseline isometric torque (in newton millimeter per kilogram of body weight (Nmm/Kg)) versus frequency (in hertz (Hz)) relationship in rat TA muscles prior to surgery to mimic a 20% by mass VML injury. Data is provided for a group of five animals selected to be given the VML injury and to have no repair performed (NR, circles) and a group of six rats to be given the VML injury and have repair performed via administration of a hyaluronic acid-heparin hydrogel (HyA, squares). Data is presented as the mean±SEM. Dotted lines indicate 95% confidence limits (Cl) of sigmoidal interpolation.

FIG. 2B is a graph showing the isometric torque (in Nmm/Kg) versus frequency (in Hz) relationship in rat TA muscles four weeks after surgery to mimic a 20% by mass VML injury. Data is provided for a group of five animals given the VML injury but that had no repair performed (NR, circles) and a group of six rats given the VML injury and that had repair performed via administration of a hyaluronic acid-heparin hydrogel (HyA, squares). Data is presented as the mean±SEM. Dotted lines indicate 95% Cl of sigmoidal interpolation.

FIG. 2C is a graph showing the isometric torque (in Nmm/Kg) versus frequency (in Hz) relationship in rat TA muscles eight weeks after surgery to mimic a 20% by mass VML injury. Data is provided for a group of five animals given the VML injury but that had no repair performed (NR, circles) and a group of six rats given the VML injury and that had repair performed via administration of a hyaluronic acid-heparin hydrogel (HyA, squares). Data is presented as the mean±SEM. Dotted lines indicate 95% Cl of sigmoidal interpolation.

FIG. 2D is a graph showing the isometric torque (in Nmm/Kg) versus frequency (in Hz) relationship in rat TA muscles twelve weeks after surgery to mimic a 20% by mass VML injury. Data is provided for a group of five animals given the VML injury but that had no repair performed (NR, circles) and a group of six rats given the VML injury and that had repair performed via administration of a hyaluronic acid-heparin hydrogel (HyA, squares). Data is presented as the mean±SEM. Dotted lines indicate 95% Cl of sigmoidal interpolation.

FIG. 2E is a graph illustrating the equivalence of the mean peak baseline tetanic contration force resulting from peroneal nerve stimulation and measured with a footplace force transducer in all treatment groups described in FIG. 2A. The graph shows the peak baseline isometric torque (in Nmm/Kg) in rat TA muscles prior to surgery to mimic a 20% by mass VML injury. Data is provided for a group of five animals given the VML injury but that had no repair performed (NR, grey shaded bar) and a group of six rats given the VML injury and that had repair performed via administration of a hyaluronic acid-heparin hydrogel (HyA, black shaded bar).

FIG. 2F is a bar graph of the peak isometric torque (in Nmm/Kg) in rat TA muscles four, eight, or twelve weeks after surgery to mimic a 20% by mass VML injury as indicated on the x-axis. Data is provided for a group of five animals given the VML injury but that had no repair performed (NR, grey shaded bars) and a group of six rats given the VML injury and that had repair performed via administration of a hyaluronic acid-heparin hydrogel (HyA, black shaded bars). Data is presented as the mean±SEM. Mean±SEM of baseline torque of all animals is represented by the dotted line/shaded region. * significantly different at p<0.05, ** significantly different at p<0.01, using Sidak's post-hoc after performing Two-Way ANOVA.

FIG. 2G is a bar graph of the isometric torque (presented as a percentage (%) of the initial maximum pre-injury (baseline) torque)) in rat TA muscles four, eight, or twelve weeks after surgery to mimic a 20% by mass VML injury as indicated on the x-axis. Data is provided for a group of five animals given the VML injury but that had no repair performed (NR, grey shaded bars) and a group of six rats given the VML injury and that had repair performed via administration of a hyaluronic acid-heparin hydrogel (HyA, black shaded bars). ** significantly different at p<0.01 using Sidak's post-hoc after performing Two-Way ANOVA.

FIG. 2H is a graph of the body weight (in grams (g)) in rats prior to (baseline (BL)), four, eight, or twelve weeks after surgery to mimic a 20% by mass VML injury as indicated on the x-axis. Data is provided for a group of five animals given the VML injury but that had no repair performed (NR, circles) and a group of six rats given the VML injury and that had repair performed via administration of a hyaluronic acid-heparin hydrogel (HyA, squares). Data is presented as the mean±standard error of the mean (SEM). No differences were observed among groups at each time point according to Two-Way ANOVA p=0.05.

FIG. 3A is a schematic representation of a TA muscle transverse section indicating where measurements are made for the data presented for outer muscle in FIGS. 3B and 3C.

FIG. 3B is a graph showing the median of fiber cross-sectional area (FCSA) distribution (in square micrometers (μm²)) in the outer portion of the TA muscle twelve weeks after surgery to mimic a 20% by mass VML injury. Data is provided for a group of five animals given the VML injury but that had no repair performed (NR) and a group of six rats given the VML injury and that had repair performed via administration of a hyaluronic acid-heparin hydrogel (HyA). Data is also provided for a group of 11 control rats (CTRL) that did not have the VML surgery. Data are presented as the median±minimum and maximum. * significantly different at p<0.05, ** significantly different at p<0.01, using Sidak's post-hoc after performing one-way ANOVA.

FIG. 3C is a graph showing the fiber cross-sectional area (FCSA) frequency distribution (presented as a percentage (%)) of FCSA of different area (in square micrometers (μm²)) in the outer portion of the TA muscle twelve weeks after surgery to mimic a 20% by mass VML injury. Data is provided for a group of five animals given the VML injury but that had no repair performed (NR, shaded circles) and a group of six rats given the VML injury and that had repair performed via administration of a hyaluronic acid-heparin hydrogel (HyA, shaded squares). Data is also provided for a group of 11 control rats (CTRL, unfilled triangles) that did not have the VML surgery. Data are presented as the median±minimum and maximum.

FIG. 3D is a schematic representation of a TA muscle transverse section indicating where measurements are made for the data related to inner muscle presented in FIGS. 3E and 3F.

FIG. 3E is a graph showing the median of FCSA distribution (in square micrometers (μm²)) in the inner TA muscle twelve weeks after surgery to mimic a 20% by mass VML injury. Data is provided for a group of five animals given the VML injury but that had no repair performed (NR) and a group of six rats given the VML injury and that had repair performed via administration of a hyaluronic acid-heparin hydrogel (HyA). Data is also provided for a group of 11 control rats (CTRL) that did not have the VML surgery. Data are presented as the median±minimum and maximum.

FIG. 3F is a graph showing the FCSA frequency distribution (presented as a percentage (%)) of FCSA of different area (in square micrometers (μm²)) in the inner portion of the muscle twelve weeks after surgery to mimic a 20% by mass volumetric muscle loss (VML) injury. Data is provided for a group of five animals given the VML injury but that had no repair performed (NR, shaded circles) and a group of six rats given the VML injury and that had repair performed via administration of a hyaluronic acid-heparin hydrogel (HyA, shaded squares). Data is also provided for a group of 11 control rats (CTRL, unfilled triangles) that did not have the VML surgery. Data are presented as the median±minimum and maximum.

FIG. 4 is a graph showing the quantification of the number of capillaries per individual muscle fiber in the outer and inner TA muscle in a group of six healthy/uninjured rats (CTRL) and in a group of six rats that had undergone surgery to mimic a 20% by mass VML injury and that had repair performed via administration of a hyaluronic acid-heparin hydrogel (HyA). No significant different (p<0.05 after T-test) on capillary density was observed between groups. Data is presented as the mean±SEM.

FIG. 5A is a schematic drawing showing the location and size of an exemplary surgically-created injury in the latissimus dorsi (LD) muscle of a rat to mimic VML injury. Injury is created near the spinal origin and removes a portion of the fibers that are oriented parallelly.

FIG. 5B is a photographic image showing the application of a hyaluronic acid-heparin hydrogel of the presently disclosed subject matter to an injury to a LD muscle as shown in FIG. 5A. According to an exemplary application method of the presently disclosed subject matter, a three-dimensional (3-D) printed mold is placed over the surgically created muscle defect and gel is applied inside the mold. The mold helps the hydrogel stay in place before it is solidified.

FIG. 5C is a photographic image of the surgically created muscle defect shown in FIG. 5B ten minutes after application of the hydrogel and mold removal.

FIG. 6A is a schematic drawing showing a transverse view of a surgically created muscle defect in the LD muscle of a rat used to mimic VML injury. Native muscle remains on both sides of a defect area.

FIG. 6B is a micrograph image of a H&E stained cross-sectional cut of a rat LD muscle that had undergone surgically-created muscle defect as illustrated in FIG. 6A and that had been treated via administration of a hyaluronic acid-heparin hydrogel of the presently disclosed subject matter for five days. The areas in brackets show remaining native muscle to the sides of the defect area. The areas inside the white boxes (1, 2, and 3) indicate portions of the defect area being further magnified in FIGS. 6C, 6D, and 6E.

FIG. 6C shows a further magnified portion of the image described in FIG. 6B (i.e., the area enclosed in box 1 in FIG. 6) showing fragments of hydrogel detached from the injury, presumably due to handling during muscle explant. The scale bar on the right side of the image represents 100 micrometers (μm).

FIG. 6D shows a further magnified portion of the image described in FIG. 6B (i.e., the area enclosed in box 2 in FIG. 6) showing significant cell infiltration in the hydrogel. The scale bar on the right side of the image represents 100 micrometers (μm).

FIG. 6E shows a further magnified portion of the image described in FIG. 6B (i.e., the area enclosed in box 3 in FIG. 6B) showing cell infiltration in the hydrogel. The scale bar on the right side of the image represents 100 micrometers (μm).

FIG. 7A is a graph showing the swelling ratio (compared to initial wet weight) versus time (in hours) of a hyaluronic acid-heparin hydrogel of the presently disclosed subject matter and having a weight average molecule weight 500 kilodaltons (kDa) in phosphate buffered saline (PBS) comprising 10% by volume fetal bovine serum (FBS) at 37 degrees Celsius (° C.).

FIG. 7B is a graph showing the swelling ratio (compared to lyophilized weight) versus time (in hours) of a lyophilized patch prepared from a hyaluronic acid-heparin hydrogel of the presently disclosed subject matter and having a weight average molecule weight 500 kDa in PBS comprising 10% by volume FBS at 37° C.

FIG. 7C is a series of photographic images of (top) the lyophilized patch described in FIG. 7B just prior to swelling in PBS comprising 10% by volume FBS, (middle) the same patch after four hours of swelling in PBS comprising 10% by volume FBS at 37° C.; and (bottom) the same patch after 24 hours of swelling in PBS comprising 10% by volume FBS at 37° C. The patch starts out opaque in the dry state (top), but gradually becomes transparent as it wets and swells.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. Although any methods, devices, and materials similar or equivalent to those described herein can be used in the practice or testing of the presently disclosed subject matter, representative methods, devices, and materials are now described. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a hydrogel polymer” includes a plurality of such hydrogel polymers, and so forth.

Unless otherwise indicated, all numbers expressing quantities of size, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size, diameter, thickness, weight, concentration, time, or percentage is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4).

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “substantially,” when referring to a value, an activity, or to an amount of a composition, mass, weight, temperature, time, volume, concentration, percentage, etc., is meant to encompass variations of in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions. For example, a subject is “substantially treated” when the condition to be treated is at least 60%, or at least 75%, or at least 80%, or at least 85%, or at least 90%, or at least 95%, and, in certain cases, at least 99% treated or resolved.

As used herein, a “subject in need thereof” is a patient, animal, mammal, or human, who will benefit from the methods and/or treatments of this disclosure.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The terms “biological cell” and “cell” can be used interchangeably herein and can refer to a differentiated or undifferentiated from a living organism. Thus, cells include differentiated and undifferentiated animal or plant cells. Exemplary cells include, but are not limited to, stem cells, progenitor cells, and muscle-derived cells (i.e., cells derived from muscle tissue or cultured muscle tissue or cells).

The term “growth factor” as used herein refers to any endogenous factor that modulates (e.g., increases or decreases) the growth, proliferation, survival, differentiation (e.g., a factor that promotes differentiation, a factor that inhibits differentiation, a factor that reverses differentiation, e.g., a de-differentiation factor, a pluripotency factor, etc.), and/or function of a cell in contact with the presently disclosed hydrogel matrices. In some embodiments, the term “growth factor” is used broadly to encompass endogenous factors (i.e., growth factors present in a subject being treated and not administered during a muscle injury repair procedure) that modulate the growth, proliferation, survival, differentiation, and/or function of a cell. Growth factors include but are not limited to: a colony stimulating factor, (e.g., a granulocyte colony stimulating factor (G-CSF), granulocyte-monocyte colony stimulating factor, macrophage colony stimulating factor, megakaryocyte colony stimulating factor, and the like), a growth hormone (e.g., a somatotropin, a human growth hormone, and the like), an interleukin (e.g., IL-1, IL-2, including, e.g., IL-3, IL-4, IL-5, IL-6, IL-7, IL-8, IL-9, etc.), a growth factor, a stem cell factor, keratinocyte growth factor, an acidic fibroblast growth factor, a basic fibroblast growth factor, a hepatocyte growth factor (HGF), a chemokine, an angiogenic agent (e.g., vascular endothelial growth factor (VEGF)), an EGF (epidermal growth factor), a receptor tyrosine kinase ligand, thrombolytic agent, an atrial natriuretic peptide, bone morphogenic protein, thrombopoietin, relaxin, glial fibrillary acidic protein, follicle stimulating hormone, a human alpha-1 antitrypsin, a leukemia inhibitory factor, a transforming growth factor, a tissue factor, an insulin-like growth factor and/or its binding proteins, a luteinizing hormone, a follicle stimulating hormone, a macrophage activating factor, tumor necrosis factor, a neutrophil chemotactic factor, a nerve growth factor, a tissue inhibitor of metalloproteinases, a vasoactive intestinal peptide, angiogenin, angiotropin, fibrin, hirudin, a leukemia inhibitory factor, a Wnt signaling ligand (e.g., Wnt, norrin, R-spondin, etc.), a Wnt signaling inhibitor (e.g, WIF (Wnt inhibitory factor), sFRP (Secreted Frizzled Related Protein), Dkk (Dickkopf), Notum, and the like), a Notch or Notch ligand protein, a receptor tyrosine kinase ligand, a hedgehog (HH) pathway ligand (e.g., HH), and a transforming growth factor-β (TGF-β).

The terms “nanoparticle” and “nanoparticulate” as used herein refer to a structure having at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm).

The terms ““microparticle” and “microparticulate” as used herein refer to a structure having at least one region with a dimension (e.g., length, width, diameter, etc.) of less than about 1,000 μm. In some embodiments, the dimension is smaller (e.g., about 500 μm, about 250 μm, about 200 μm, about 150 μm, about 125 μm, about 100 μm, about 80 μm, about 70 μm, about 60 μm, about 50 μm, about 40 μm, about 30 μm, about 20 μm, or about 10 μm).

The micro- or nanoparticles can have any three-dimensional shape. In some embodiments, the particles are approximately spherical. In some embodiments, the particles are disc, cube or rod shaped. In some embodiments, the particles are irregularly shaped.

The term “diameter” is art-recognized and is used herein to refer to either the physical diameter or the hydrodynamic diameter. The diameter of an essentially spherical particle can refer to the physical or hydrodynamic diameter. As used herein, the diameter of a non-spherical particle can refer to the largest linear distance between two points on the surface of the particle. When referring to multiple particles, the diameter of the particles typically refers to the average diameter of the particles. Particle diameter can be measured using a variety of techniques in the art including, but not limited to, dynamic light scattering (DLS).

The term “substantially two-dimensional material” as used herein refers to a material that is at least 10, 25, 50, 100, 250, 500, or 1000 times wider and/or longer than it is thick. Thus, the “substantially two-dimensional material” can be a sheet-like material or a thin wafer or disc.

As used herein, a “macromolecule” refers to a molecule of high relative molecular mass, the structure of which comprises the multiple repetition of units derived from molecules of low relative molecular mass, e.g., monomers and/or oligomers.

An “oligomer” refers to a molecule of intermediate relative molecular mass, the structure of which comprises a small plurality (e.g., 2, 3, 4, 5, 6, 7, 8, 9, or 10) of repetitive units derived from molecules of lower relative molecular mass.

As used herein, a “monomer” refers to a molecule that can undergo polymerization, thereby contributing constitutional units, i.e., an atom or group of atoms, to the essential structure of a macromolecule.

The terms “polymer” and “polymeric” refer to chemical structures that have repeating constitutional units (i.e., multiple copies of a given chemical substructure or “monomer unit” or “monomeric” units). As used herein, polymers can refer to groups having more than 10 repeating units and/or to groups wherein the repeating unit is other than methylene. Polymers can be formed from polymerizable monomers. A polymerizable monomer is a molecule that comprises one or more reactive moieties {e.g., siloxy ethers, hydroxyls, amines, vinylic groups (i.e., carbon-carbon double bonds), halides (i.e., Cl, Br, F, and I), carboxylic acids, esters, activated esters, and the like} that can react to form bonds with other molecules. Generally, each polymerizable monomer molecule can bond to two or more other molecules. In some cases, a polymerizable monomer will bond to only one other molecule, forming a terminus of the polymeric material. Some polymers contain biodegradable linkages, such as esters or amides, such that they can degrade overtime under biological conditions (e.g., at a certain pH present in vivo or in the presence of enzymes).

A “copolymer” refers to a polymer derived from more than one species of monomer. Each species of monomer provides a different species of monomer unit.

Polydispersity (PDI) refers to the ratio (M_{w}/Mₙ) of a polymer sample. M_{w} refers to the mass average molar mass (also commonly referred to as weight average molecular weight). Mₙ refers number average molar mass (also commonly referred to as number average molecular weight).

“Biocompatible” as used herein, generally refers to a material and any metabolites or degradation products thereof that are generally non-toxic to a biological organism, tissue, or cell, and which do not cause any significant adverse effects to the biological organism, tissue, or cell.

The terms “crosslinking agent” or “cross-linker” refer to a compound that includes at least two reactive functional groups (or groups that can be deblocked or deprotected to provide reactive functional groups), which can be the same or different. In some embodiments, the two reactive functional groups can have different chemical reactivity (e.g., the two reactive functional groups are reactive (e.g., form bonds, such as covalent bonds) with different types of functional groups on other molecules, or one of the two reactive functional groups tends to react more quickly with a particular functional group on another molecule than the other reactive functional group). Thus, the cross-linking reagent can be used to link (e.g., covalently bond) two other entities (e.g., two different polymer chains) or to link two groups on the same entity (e.g., a single polymer chain) to form a crosslinked composition.

Generally, as used herein, the term “crosslinked” refers to a composition comprising multiple bonds or linkages between two entities or comprising multiple added bonds or linkages between groups on the same entity. Crosslinked can refer to covalent crosslinking or associative crosslinking.

The term “hydrogel matrix” as used herein refers to a network of polymer chains (“hydrogel polymers”) that are water-insoluble, sometimes found as a colloidal gel in which water is the dispersion medium. Hydrogel matrices can contain over 99% water and can comprise natural or synthetic polymers, or a combination thereof. In other instances, hydrogels can contain other percentages of water. Hydrogels also possess a degree of flexibility due to their significant water content.

The term “crosslinked hydrogel matrix” as used herein refers to a a composition comprising hydrogel polymers further comprising at least one and typically more than one additional bond formed between sites on an individual hydrogel polymer chain and/or between individual hydrogel polymer chains. In some embodiments, the sites are bonded to one another via a linker group formed when a crosslinking agent bonds to two different sites on a hydrogel polymer chain or to sites on two different hydrogel polymer chains.

### II. General Considerations

Despite the well-documented capability of skeletal muscle to repair, regenerate, and remodel following injury (see Carlson and Faulkner, Med. Sci. Sports Exerc. 15, 187-198 (1983); Carson, J. Morphol. 125, 447-471 (1968); Ciciliot and Schiaffino, Curr. Phar, Des. 16, 906-914 (2010); Warren et al., J. Physiol. 582, 825-841 (2007); and White and Devor, Exerc. Sport Sci. Rev. 21, 263-295 (1993)), there remains a multitude of diseases, disorders, and traumas that result in irrecoverable loss of muscle function. Included among these traumatic injuries is volumetric muscle loss (VML). See Grogan and Hsu, J. Am. Acad. Orthop. Surg. 19 Suppl 1, S35-S37 (2011). A VML injury is characterized by such a significant degree of muscle tissue loss that it exceeds the native ability of the muscle to recover, thereby resulting in permanent cosmetic and functional deficits (see Holcomb et al., J Trauma Ing. Infect. Crit. Care 60, 397-401 (2006)) to the limbs, neck, or face. These injuries impact both the civilian and military populations, affecting thousands of individuals each year.

Current options for regenerative therapeutics for treatment of VML injury have shown a limited ability to produce functional and cosmetic recovery and are frequently associated with harvest and donor site morbidities. See Lin et al., Plast. Reconstr. Surg. 119, 2118-2126 (2007). In response to this, attention has focused on tissue engineering (TE) technologies to provide more effective treatment options for large scale muscle injuries. A common TE approach has been the implantation of decellularized extracellular matrices (ECM), such as decellularized bladder collagen scaffolds, both with (see Corona et al., Tissue Eng. Part A 18, 1213-1228 (2012); Corona et al., Biomaterials, 34, 3324-3335 (2013), Criswell et al., Biomaterials 34, 140-149 (2013); Machingal et al., Tissue Eng. Part A 17, 2291-2303 (2011); Merritt et al., Tissue Eng. Part A 16, 2871-2881 (2010); VanDusen et al., Tissue Eng. Part A 20, 2920-2930 (2014); and Williams et al., J. Tissue Eng. Regen. Med. 7, 434-442 (2013)) and without cellular components. See Merritt et al., Tissue Eng. Part A 16, 1395-1405 (2010); Perniconi et al., Biomaterials 32, 7870-7882 (2011); Sicari et al., Tissue Eng. Part A 18, 1941-1948 (2012); and Turner et al., J. Surg. Res. 176, 490-502 (2012). Several of these approaches have been evaluated in preclinical studies, with results showing that the inclusion of a cellular component generally leads to a greater functional improvement. See Corona et al., Tissue Eng. Part A 18, 1213-1228 (2012); Machingal et al., Tissue Eng. Part A 17, 2291-2303 (2011); and Corona et al., Tissue Eng. Part A 20, 705-715 (2014). However, recent clinical studies for treatment of VML injury solely with implanted ECM scaffolds have shown some evidence of functional recovery, but with limited de novo muscle tissue regeneration at the injury site.

The presently disclosed subject matter is based in part on the finding that growth factor-free and cell-free hydrogel systems can treat muscle injury (e.g., VML injury) to provide improved functional recovery and de novo muscle tissue regeneration. In some embodiments, the presently disclosed subject matter relates to the use of hydrogel systems, such as HyA-heparin hydrogels, comprising moieties that can bind and present endogenous growth factors within a matrix comprising the hydrogel (i.e., “growth factor recruitment moieties”) following administration in a subject to support functional muscle regeneration and/or repair. As described hereinbelow, a previously established and biologically relevant rat tibialis anterior (TA) VML injury model was used to evaluate the ability of application of exemplary HyA-heparin hydrogels to restore skeletal muscle function and native tissue morphology. The bioinspired hydrogel system was implanted in a VML injury model in the TA muscle. For example, as described further hereinbelow, the hydrogel system can be employed by injecting HyA and/or other hydrogel macromers into the injured muscle, where they polymerize in situ by reaction with crosslinking agents, e.g., proteolytically cleavable crosslinking peptides. Samples retrieved 12 weeks after administration of the exemplary HyA-heparin hydrogel system showed significant functional recovery, which is believed to signify a significant leftward shift compared to recovery previously observed in this injury model with any technology yet evaluated. Furthermore, volume reconstitution, muscle regeneration, and native-like vascularization were also observed to a greater extent than previously observed in this model with other technologies. See Corona et al. Tissue Eng. Part A 20, 705-715 (2014); and Passipieri et. al., Tissue Eng. Part A 23, 556-571 (2017). The retrieved tissue wet weight and median fiber cross-sectional area (FCSA) in HyA-heparin hydrogel treated animals were similar to the contralateral control TA muscle, and both parameters in those groups were significantly different (larger) than the nonrepaired (NR) group. These results are unexpected given that the presently disclosed hydrogel system contains no exogenous growth factors or cells, which are typically thought to be required to promote muscle repair. Thus, without being bound to any one theory or mechanism of action, the instant disclosure establishes that hydrogel can recruit and retain stem and progenitor cells to the injury site, where the hydrogel then captures endogenously synthesized growth factors and presents them from the solid phase within the microenvironment. In this way, the hydrogel creates a more favorable environment for tissue regeneration to occur, potentiating repair.

As further described below, the presently disclosed cell- and exogenous growth factor-free hydrogel system has also been injected into the full-thickness Latissimus Dorsi (LD) injury model, a significantly larger and more challenging muscle injury. After delivery, but prior to complete gelation, the solution of hydrogel components had the required viscosity to be retained within the injury site. For example, after approximately 10 minutes, the hydrogel appeared stable, with good adhesion apparent between the hydrogel and surrounding muscle. Furthermore, the hydrogel had the stability to be sutured in place, to ensure that it would be retained in the injury site even after the animal was awake and mobile.

Swelling of the hydrogel was observed in samples retrieved 1 hour and 5 days after implantation. This is useful in that it can ensure complete filling of the wound volume, as well as good contact between the hydrogel and host tissue. Furthermore, it implies that the hydrogel acts like a sponge and soaks up exudate from the injured muscle, potentially creating an environment rich in endogenous growth factors and potentiating repair as in the TA injury model.

Additional features of the presently disclosed subject matter have positive indications for use in muscle repair even in austere environments (e.g., field use, such as in the battlefield). For instance, the hydrogel can be lyophilized to yield a freeze-dried sheet of the hydrogel matrix. The freeze-dried sheet can be cut or otherwise formed into wafers or discs of any suitable size and shape for facile transport, storage, and on-demand application. The freeze-dried sheet is also suturatable. The lyophilized hydrogel can also be prepared as a powder (e.g., by grinding a lyophilized sheet of the hydrogel or via initial preparation of the gel in particle form (e.g., via emulsion polymerization)). The powder form is also easily stored and transported. Once administered to a wound site or placed in contact with an aqueous environment, the lyophilized hydrogel shows robust swelling, indicating that once inserted in a muscle injury site the lyophilized hydrogel form can rapidly expand to fill injury site. It can also become complexed with clotting blood to form a provisional matrix embodying both a biological fibrin clot and the HyA-heparin hydrogel particles.

### III. Methods and Compositions for the Treatment of Muscle Injury

Accordingly, in some embodiments, the presently disclosed subject matter provides a method of treating muscle injury, e.g., VML injury, the method comprising: (a) providing a subject in need of muscle injury treatment (e.g., a subject in need of VML treatment); and (b) administering to the subject (i.e., at a muscle injury site, such as a VML injury site) a regenerative hydrogel system, wherein said regenerative hydrogel system comprises a growth factor recruitment moiety (e.g., a glycosaminoglycan, such as heparin or a derivative or copolymer thereof) and wherein the regenerative hydrogel system, at the time of administration, is free of growth factors and biological cells; whereby the muscle injury, e.g., VML injury, in the subject is substantially treated. The hydrogel system can be administered as a mixture of hydrogel polymers and other components that further polymerize and/or crosslink (i.e., “gel”) to form a crosslinked hydrogel matrix after administration. Alternatively, the hydrogel system can be administered as a lyophilized sheet or powder of a crosslinked hydrogel matrix material.

For instance, in some embodiments, the regenerative hydrogel system comprises a plurality of hydrogel matrix precursors, wherein said precursors comprise: (i) one or more hydrogel polymers; (ii) a growth factor recruitment moiety (e.g., heparin or a derivative thereof); and (iii) crosslinking agent, e.g., a proteolytically cleavable cross-linker peptide. Thus, in some embodiments, the administering of step (b) comprises administering the plurality of hydrogel matrix precursors to a muscle injury site in the subject, whereby the precursors form a crosslinked hydrogel matrix in situ in the muscle injury site, wherein said crosslinked hydrogel matrix comprises one or more hydrogel polymers, wherein said one or more hydrogel polymers comprises a growth factor recruitment moiety-conjugated hydrogel polymer (e.g., a heparin-conjugated hydrogel polymer), and wherein the crosslinking agent (e.g., the proteolytically cleavable cross-linker peptide) links one of the one or more hydrogel polymers to another of the one or more hydrogel polymers. In some embodiments, the presently disclosed hydrogel system can form a crosslinked gel using a Michael Addition reaction, which can occur at physiological pH, temperatures, and divalent cation concentrations. It also produces no cytotoxic by-products. In some embodiments, the precursors can be administered essentially simultaneously, e.g., by use of a dual barreled syringe wherein one barrel (i.e. a first barrel) is filled with the one or more hydrogel polymers and the growth factor recruitment moiety (e.g., a derivatived heparin or other growth factor recuirment moiety capable of covalent bonding to one or the one or more hydrogel polymers) and wherein the other barrel (i.e. a second barrel) of the syringe is filled with one or more crosslinking agents (e.g., the proteolytically cleavable cross-linker peptide). Alternatively, the precursors can be mixed first and and then administered to the injury site. In some embodiments, the precursors can be mixed and administered to the injury site via syringe prior to gelation. For example, the exemplarily HyA hydrogels of the presently disclosed subject matter can have an approximately 5-10 minute working time prior to gelation. During this interval, it can be injected, e.g., through 18-28-gauge needle. Thus, in some embodiments, the precursors can be mixed and administered to the injury site within about 10 minutes or less or within about 5 minutes of less of mixing.

In some embodiments, to aid in positioning of the hydrogel, a mold can be placed in the muscle injury site prior to the administration of the hydrogel precursors. The mold can be, for example, a plastic mold comprising a plastic that is non-reactive to the hydrogel and the crosslinking agent (e.g., the cross-linker peptide). In some embodiments, the mold is a three-dimensionally printed mold, e.g., designed to fit a particular injury site in a subject in need thereof. The mold can then be removed from the injury site after the hydrogel precursors are allowed to crosslink/gel for a period of time (e.g., about 15 minutes or less, about 10 minutes or less, or about 5 minutes of less), leaving behind the crosslinked/gelled hydrogel in a desired position in the injury site. If suitable, the injury site can then be closed (e.g., stitched closed) and/or covered with a suitable dressing or skin graft. If desired, the crosslinked hydrogel can be sutured in place, e.g., prior to closure of the injury site. Thus, in some embodiments, the administering comprises placing a removable mold in the muscle injury site prior to the administering of step (b) to define an area where the crosslinked hydrogel matrix is to be formed and where the plurality of hydrogel precursors are to be administered; and wherein the method further comprises removing the mold from the injury site following the administration of step (b). In some embodiments, the mold is removed from the injury site about 5 to 15 minutes after the administering of step (b) (e.g., about 5, 6, 7, 8, 9, 10, 11, 12, 13, 14, or 15 minutes after the administering of step (b)). In some embodiments, the method further comprises suturing the crosslinked hydrogel matrix in place in the muscle injury site.

In some embodiments, the regenerative hydrogel system comprises a lyophilized crosslinked hydrogel matrix material, said material comprising: (i) one or more hydrogel polymers, (ii) a growth factor recruitment moiety (e.g., a glycosamino glycan, such as heparin), wherein said growth factor recruitment moiety is conjugated to one or more hydrogel polymer; and (iii) a crosslinking agent, e.g., a proteolytically cleavable cross-linker peptide, wherein the crosslinking agent links one of the one or more hydrogel polymers to another of the one or more hydrogel polymers. The lyophilized crosslinked hydrogel matrix material can be administered to the muscle injury site in any suitable form. In some embodiments, the lyophilized crosslinked hydrogel matrix can be administered as a substantially two-dimensional material (e.g., a sheet or a disc or wafer cut from a larger sheet) or as a powder, e.g., comprising nanoparticles and/or microparticles, of the lyophilized crosslinked hydrogel matrix material. Sheets of the lyophilized material can be provided in rolls for facile transport and/or storage.

The substantially two-dimensional material can have any suitable shape, such as a disc (i.e., a circular or oval sheet); as a rectangular or square sheet; as another regularly shaped sheet, such as, but not limited to a triangular, parallelogram, semicircular, hexagonal, pentagonal, diamond, or octagonal sheet; or as an irregularly shaped sheet (e.g., cut from a larger sheet to have a shape based on the shape of a particular muscle injury site). The substantially two-dimensional material can be prepared by forming a hydrogel in a mold or on a suitable support (e.g., in a petri dish). Once the hydrogel is crosslinked/gelled, it can be lyophilized, such as via freeze-drying or another low temperature process for removing solvent, e.g., water). In some embodiments, the substantially two-dimensional material can have a thickness of between about 10 mm and about 20 mm (e.g., about 10, 11, 12, 13, 14, 15, 16, 17, 18, 19, or 20 mm). In some embodiments, the substantially two-dimensional material comprising the lyophilized crosslinked hydrogel matrix material can have a thickness of between about 1 mm and about 10 mm (e.g. about 1, 2, 3, 4, 5, 6, 7, 8, 9, or 10 mm). In some embodiments, more than one two-dimensional lyophilized hydrogel material can be administered to a muscle injury site, either stacked upon one another or side-by-side. In some embodiments, the lyophilized hydrogel material can be cut, for example, to match a muscle dimension after accounting for swelling of the lyophilized material. The lyophilized crosslinked hydrogel can also be administered in the form of a powder (e.g. which can be poured into a muscle injury site). The powder can be provided by grinding or crushing a lyophilized sheet of the crosslinked hydrogel. Alternatively, the powder can be provided by forming the crosslinked hydrogel via emulsion polymerization to provide crosslinked hydrogel particles that can then be lyophilized. As the lyophilized powder can swell once in the muscle injury site, the amount of powder to be poured into the muscle injury site can be substantially less than that required to fill the site. For example, in some embodiments, powder is poured into the wound so that if fills about 1% to about 50% of the volume of the wound, or preferably about 25% or less, 10% or less or about 5% or less of the volume of the wound. In some embodiments, a combination of a substantially two-dimensional material and a powder of the lyophilized hydrogel can be administered in combination.

Thus, in some embodiments, the lyophilized crosslinked hydrogel matrix material is administered to a muscle injury site as a substantially two-dimensional material (e.g., a sheet) comprising the lyophilized crosslinked hydrogel matrix material. In some embodiments, the method further comprises suturing the substantially two-dimensional material (e.g., the sheet) in place in the muscle injury site. In some some embodiments, the lyophilized crosslinked hydrogel matrix material is administered to a muscle injury site in the subject in powder form.

Regardless of whether the regenerative hydrogel system is prepared in situ in an injury site or administered in lyophilized form, any suitable hydrogel polymer or polymers can be used. In some embodiments, one or more hydrogel polymers or copolymers can be formed from monomers selected from the group including, but not limited to, lactic acid, glycolic acid, acrylic acid, 1-hydroxyethyl methacrylate (HEMA), ethyl methacrylate (EMA), propylene glycol methacrylate (PEMA), acrylamide (AAM), N-vinylpyrrolidone, methyl methacrylate (MMA), glycidyl methacrylate (GDMA), glycol methacrylate (GMA), ethylene glycol, fumaric acid, and the like. The hydrogel can be homopolymeric or can comprise copolymers prepared from two or more different monomers. In some embodiments, the one or more hydrogel polymers include a temperature-sensitive hydrogel polymer. One exemplary temperature-sensitive hydrogel polymer is an interpenetrating hydrogel network of poly(N-isopropylacrylamide) and poly(acrylic acid), which is a copolymer that swells upon an increase in temperature (e.g., an increase in temperature to about normal body temperature, i.e., about 37° C.). In some embodiments, one or more hydrogel polymers include, but are not limited to, poly(N-isopropylacrylamide) (pNIPAAm); poly(N-isopropylacrylamide-co-acrylic acid); hyaluronic acid or hyaluronate; crosslinked hyaluronic acid or hyaluronate; pHEMA; or copolymers of p(NIPAAm)-based sIPNs and other hydrogel sIPNs (semi-interpenetrating networks). In some embodiments, the hydrogel polymer is a hyaluronic acid (HyA) polymer. In some embodiments, the hydrogel polymer is an acrylated hyaluronic acid (HyA) polymer.

In some embodiments, one of the one or more of the hydrogel polymers can be conjugated to a cell-adhesion moiety, e.g., a moiety that provides for binding to a cell-surface receptor. In some embodiments, the cell-adhesion moiety is a cell adhesion ligand that provides for binding to a cell-surface receptor on the surface of a cell. In some embodiments, the cell-binding moiety is a cell adhesion peptide. Thus, in some embodiments, the one or more hydrogel polymers further comprise a hydrogel polymer conjugated to a cell adhesion peptide.

Any suitable cell adhesion peptide can be used. In some embodiments, the cell adhesion peptide can bind an integrin. In some embodiments, the cell adhesion peptide can bind α5β1 and or αvβ3 integrin.

In some embodiments, the cell adhesion molecule can promote angiogenesis.

In some embodiments, the cell adhesion peptide has a length of 40 amino acids or less, 35 amino acids or less, 30 amino acids or less, 25 amino acids or less, or 20, 19, 18, 17, 16, 15, 14, 13, 12, 11, 10, 9, 8, 7, 6, or 5 amino acids or less. For example, in some embodiments, the cell adhesion peptide has a length of from about 3 amino acids to about 40 amino acids, e.g., from about 3 amino acids to about 5 amino acids, from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, or from about 35 amino acids to about 40 amino acids.

In some embodiments, the concentration of cell adhesion peptide in the hydrogel can range from about 50 μM to about 500 μM, e.g., from about 50 μM to about 75 μM, from about 75 μM to about 100 μM, from about 100 μM to about 125 μM, from about 125 μM to about 150 μM, from about 150 μM to about 200 μM, from about 200 μM to about 250 μM, from about 250 μM to about 300 μM, from about 300 μM to about 350 μM, from about 350 μM to about 400 μM, from about 400 μM to about 450 μM, or from about 450 μM to about 500 μM.

In some embodiments, the cell adhesion peptide is an Arg-Gly-Asp (RGD) peptide (i.e., a peptide that contains the amino acid sequence RGD). A suitable RGD peptide comprises the amino acid sequence: CGGNGEPRGDTYRAY (SEQ ID NO:2). Also suitable for use are peptides comprising the amino acid sequence FHRRIKA (SEQ ID NO:3). Also suitable for use are the peptides acetyl-CGGNGEPRGDTYRAY-NH₂ (SEQ ID NO:4) and acetyl-CGGFHRRIKA-NH₂ (SEQ ID NO:5). Other suitable peptides are shown in Table 1, below.

TABLE 1
Exemplary Cell Adhesion Peptides.

| Peptide | SEQ ID NO: |
|---|---|
| CGGNGEPRGDTYRAY | SEQ ID NO: 2 |
| CEPRGDTYRAYG | SEQ ID NO: 6 |
| CGGGEAPRGDVY | SEQ ID NO: 7 |
| CCGPRGDVYG | SEQ ID NO: 8 |
| CGGVSWFSRHRYSPFAVS | SEQ ID NO: 9 |
| CGGNRWHSIYITRFG | SEQ ID NO: 10 |
| CGGTWYKIAFQRNRK | SEQ ID NO: 11 |
| CGGRKRLQVQLSIRT | SEQ ID NO: 12 |
| CGGKAFDITYVRLKF | SEQ ID NO: 13 |
| CTRKKHDNAQ | SEQ ID NO: 14 |
| VSWFSRHRYSPFAVS | SEQ ID NO: 15 |
| RNIAEIIKDI | SEQ ID NO. 16 |
| TAGSCLRKFSTM | SEQ ID NO: 17 |
| TTSWSQCSKS | SEQ ID NO: 18 |
| RYVVLPRPVCFEK | SEQ ID NO: 19 |
| EVLLI | SEQ ID NO: 20 |

In some embodiments, the cell adhesion peptide has an additional cysteine residue added to the N-terminal side of the peptide to allow for conjugation of the peptide to a second moiety (e.g., to allow for conjugation of the peptide to a hydrogel polymer). In some instances, a peptide represented by one of the sequences set forth in SEQ ID NO:2-20, can have a cysteine residue added to the N terminal side of the sequence. In some embodiments, the cell adhesion peptide has the amino acid sequence CGGNGEPRGDTYRAY (SEQ ID NO: 2).

Any suitable growth factor recruitment moiety can be used. In some embodiments, the growth factor recruitment moiety is a polysaccharide. In some embodiments, the growth factor recruitment moiety is glycosaminoglycan. In some embodiments, the growth factor recruitment moiety is a heparin. In some embodiments, the heparin or other growth factor recruitment moiety is provided as a derivative (e.g., a polysaccharide derivative, a glycosaminoglycan derivative, a heparin derivative, a peptide mimetic of heparin, etc.) that comprises a chemical functional group capable of reacting with a group present on one of the one or more hydrogel polymers and/or a crosslinking agent used in the regenerative hydrogel system (i.e., when the heparin or other growth factor recruitment moiety is a precursor of a crosslinked hydrogel matrix material). In some embodiments, e.g., when the growth factor recruitment molecule comprises a polymer (e.g., heparin), the growth factor recruitment moiety is provided as a copolymer with one of the one or more hydrogel polymers. Thus, in some embodiments, the growth factor recruitment moiety (e.g., heparin) is provided already conjugated (e.g., covalently conjugated) to one of the hydrogel polymers. In some embodiments, the growth factor recruitment moiety is heparin or a derivative or copolymer thereof. In some embodiments, the heparin derivative is a thiolated heparin. In some embodiments, the heparin is a high molecular weight heparin (HMWH) or a derivative or copolymer thereof. In some embodiments, the heparin (e.g., the HMWH) has a weight average molecular weight (MW_{w}) of between about 6 kilodaltons (kDa) and about 12 kDa. In some embodiments, the HMWH can have a MW_{w} of about 8 kDa or more, about 9 kDa or more, about 10 kDa or more, about 11 kDa or more, or about 12 kDa or more. In some embodiments, the heparin is a HMWH having a MW_{w} of between about 8 kDa and about 12 kDa.

In some embodiments, the weight percent of the growth factor recruitment moiety in the hydrogel system (e.g., the crosslinked hydrogel matrix) can range from 0.01 weight % to about 1 weight %, e.g., 0.01 weight %, 0.02 weight %, 0.03 weight %, 0.04 weight %, 0.05 weight %, from 0.05 weight % to about 0.1 weight %, from about 0.1 weight % to about 0.25 weight %, from about 0.25 weight % to about 0.5 weight %, from about 0.5 weight % to about 0.75 weight %, or from about 0.75 weight % to 1 weight %. In some embodiments, the weight percent is between about 0.01 wt % and about 0.03 wt %. In some embodiments, the weight percent is about 0.03 wt %.

In some embodiments, the one or more hydrogel polymers can be crosslinked using any suitable crosslinking agent. In some embodiment, the crosslinking agent comprises a peptide, e.g., a proteolytically cleavable crosslinking peptide. Proteolytically cleavable crosslinker polypeptides have been previously described. See, e.g., Kim and Healy, Biomacromolecules 4, 1214 (2003). Such proteolytically cleavable cross-linker polypeptides can provide for the remodeling of the hydrogel cell matrix. Examples of proteolytically cleavable cross-linker polypeptides can include, but are not limited to, a matrix metalloproteinase (MMP) cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For instance, the cleavage sequence of MMP-9 is Pro-X-X-Hy (wherein, X represents an arbitrary residue; Hy, a hydrophobic residue) (SEQ ID NO:21), e.g., Pro-X-X-Hy-(Ser/Thr) (SEQ ID NO:22), Pro-Leu/Gln-Gly-Met-Thr-Ser (SEQ ID NO:23), or Pro-Leu/Gln-Gly-Met-Thr (SEQ ID NO:24). Another example of a protease cleavage site is a plasminogen activator cleavage site, e.g., a uPA or a tissue plasminogen activator (tPA) cleavage site. Examples of cleavage sequences of uPA and tPA include sequences comprising Val-Gly-Arg. Another example is a thrombin cleavage site, e.g., CGLVPAGSGP (SEQ ID NO:25). Additional suitable linkers comprising protease cleavage sites include linkers comprising one or more of the following amino acid sequences: 1) SLLKSRMVPNFN (SEQ ID NO:26) or SLLIARRMPNFN (SEQ ID NO:27), cleaved by cathepsin B; SKLVQASASGVN (SEQ ID NO:28) or SSYLKASDAPDN (SEQ ID NO:29), cleaved by an Epstein-Bar virus protease; RPKPQQFFGLMN (SEQ ID NO:30) cleaved by MMP-3 (stromelysin); SLRPLALWRSFN (SEQ ID NO:31) cleaved by MMP-7 (matrilysin); SPQGIAGQRNFN (SEQ ID NO:32) cleaved by MMP-9; DVDERDVRGFASFL (SEQ ID NO:33) cleaved by a thermolysin-like MMP; SLPLGLWAPNFN (SEQ ID NO:34) cleaved by matrix metalloproteinase 2 (MMP-2); SLLIFRSWANFN (SEQ ID NO:35) cleaved by cathespin L; SGVVIATVIVIT (SEQ ID NO:36) cleaved by cathespin D; SLGPQGIWGQFN (SEQ ID NO:37) cleaved by matrix metalloproteinase 1 (MMP-1); KKSPGRVVGGSV (SEQ ID NO:38) cleaved by urokinase-type plasminogen activator; PQGLLGAPGILG (SEQ ID NO:39) cleaved by membrane type 1 matrixmetalloproteinase (MT-MMP); HGPEGLRVGFYESDVMGRGHARLVHVEEPHT (SEQ ID NO:40) cleaved by stromelysin 3 (or MMP-11), thermolysin, fibroblast collagenase and stromelysin-1; GPQGLAGQRGIV (SEQ ID NO:41) cleaved by matrix metalloproteinase 13 (collagenase-3); GGSGQRGRKALE (SEQ ID NO:42) cleaved by tissue-type plasminogen activator (tPA); SLSALLSSDIFN (SEQ ID NO:43) cleaved by human prostate-specific antigen; SLPRFKIIGGFN (SEQ ID NO:44) cleaved by kallikrein (hK3); SLLGIAVPGNFN (SEQ ID NO:45) cleaved by neutrophil elastase; and FFKNIVTPRTPP (SEQ ID NO:46) cleaved by calpain (calcium activated neutral protease); and CQPQGLAKC (SEQ ID NO:1) cleaved by matrix metalloproteinase 13 (MMP-13). Additional sequences that can be cleaved by an MMP (e.g., MMP-13, MMP-2, and MMP-9) include GPLGMHGK (SEQ ID NO: 47) and GPLGLSLGK (SEQ ID NO: 48). See Jha et al., Biomaterials 89, 136-147 (2016).

In some embodiments, the proteolytically cleavable cross-linker polypeptide can be cleaved by an MMP. In some embodiments, the proteolytically cleavable cross-linker polypeptide can be cleaved by MMP-13, MMP-2, or MMP-9. In some embodiments, the proteolytically cleavable cross-linker comprises a cysteine at the N-terminus and at the C terminus. In some embodiments, the proteolytically cleavable cross-linker polypeptide includes the amino acid sequence CQPQGLAKC (SEQ ID NO:1).

The swelling ratio of the hydrogel can be controlled by the amount of crosslinking agent used, while the in vivo degradation rate of the hydrogel can be controlled by the selection of particular crosslinking agent. For example, in some embodiments, the in vivo degradation rate of the hydrogel can be controlled by the use of a particular proteolytically cleavable crosslinker peptide that is designed to be cleaved by enzymes present or anticipated to be present in the environment of the muscle injury site to be treated. In some embodiments, the crosslinking density can be defined as moles of thiol on the peptide cross-linker compared to moles of acrylate groups on AcHyA hydrogel polymers. In some embodiments, the crosslinking density can be between about 25% and about 100%.

The administration of the regenerative hydrogel system can provide improved muscle recovery compared to untreated muscle injury, despite the lack of exogenous growth factors and biological cells in the system. In some embodiments, the improved muscle recovery is improved compared to that expected if the injury had been treated using a cell-seeded acellular ECM, a cell-seeded hydrogel system and/or a hydrogel system administered with exogenous growth factors. In some embodiments, the improved muscle recovery comprises one or more of increased muscle mass, increased muscle volume, improved muscle vascularization (e.g., a higher number of capillaries per muscle fiber), native-like muscle vascularization, and improved muscle function as compared to an untreated injury or an injury treated using a different system. In some embodiments, improved muscle mass and/or volume can be correlated to improved muscle function (e.g., improved muscle force of contraction). In some embodiments, the improved muscle recovery occurs within about 8 to 12 weeks of administration (e.g., about 8 weeks, about 9 weeks, about 10 weeks, about 11 weeks, or about 12 weeks) of the regenerative hydrogel system. In some embodiments, administration of the regenerative hydrogel system provides increased muscle mass compared to non-treated injury within about 8 to 12 weeks of administration. In some embodiments, increased muscle mass occurs within about 8 weeks of administration. In some embodiments, administration provides a recovery of muscle function of a VML injury within about 8 weeks of administration of the regenerative hydrogel system, wherein the recovery of muscle function is between about 50% and about 99%. In some embodiments, recovery of muscle function is about 80% or more (e.g., about 80%, about 85%, about 87.5%, about 89%, about 90%, or about 91% or more) compared to the maximum potential muscle function recovery. However, particularly for larger muscles (e.g., human leg muscles), recovery of less than 50% can still be of value. Thus, in some embodiments, the recovery of muscle function is between about 20% and about 99%.

In some embodiments, the subject of the presently disclosed subject matter is a human. However, subjects to be treated by the methods of the presently disclosed subject matter include both human subjects and other animal subjects (particularly mammalian subjects such as dogs and cats) for veterinary purposes.

More particularly, the terms “subject”, “patient” or “recipient” as used herein can be used interchangeably and can refer to a member of any invertebrate or vertebrate species. Accordingly, the term “subject” is intended to encompass any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals)), and all Orders and Families encompassed therein.

The compositions and methods of the presently disclosed subject matter are particularly useful for warm-blooded vertebrates. Thus, the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, hamsters, guinea pigs, and rabbits), marsupials, and horses. Also provided is the use of the disclosed methods and compositions on birds, including those kinds of birds that are endangered, kept in zoos or as pets (e.g., parrots, cockatiels and the like), as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As described hereinabove, the presently disclosed methods can, in some embodiments, employ lyophilized versions of the regenerative hydrogel system that is free of cells and growth factors prior to use. Thus, in some embodiments, the presently disclosed subject matter provides such lyophilized crosslinked hydrogel matrix materials. In some embodiments, the lyophilized crosslinked hydrogel matrix material comprises: (i) one or more hydrogel polymers, (ii) a growth factor recruitment moiety (e.g., a glycosaminoglycan, such as heparin), wherein said growth factor recruitment moiety is conjugated to one or more hydrogel polymer; and (iii) a crosslinking agent, e.g., a proteolytically cleavable cross-linker peptide, wherein the crosslinking agent, e.g., the proteolytically cleavable cross-linker peptide, links one of the one or more hydrogel polymers to another of the one or more hydrogel polymers. The one or more hydrogel polymers can comprise any of the hydrogel polymers described above, or a combination thereof. In some embodiments, the one or more hydrogel polymers comprise an acrylated hyaluronic acid polymer (HyA).

In some embodiments, the growth factor recruitment moiety is a heparin, wherein said heparin is covalently conjugated to one of the one or more hydrogel polymers (e.g., an acrylated HyA). In some embodiments, the heparin is a high molecular weight heparin (HMWH). In some embodiments, the heparin (e.g., the HMWH) has a MW_{w} of at least 6 kDa or at least 7 kDa. In some embodiments, the HMWH has a MW_{w} between about 6 kDa and about 12 kDa. In some embodiments, the HMWH has a MW_{w} of between about 8 kDa and about 12 kDa (e.g., about 8 kDa, about 9 kDa, about 10 kDa, about 11 kDa, or about 12 kDa). In some embodiments, the HMWH has a MW_{w} of about 12 kDa or more. In some embodiments, the lyophilized crosslinked hydrogel matrix comprises between about 0.01 wt % and about 0.03 wt %. of the growth factor recruitment moiety. In some embodiments, the lyophilized crosslinked hydrogel matrix comprises about 0.03 wt % of the growth factor recruitment moiety.

In some embodiments, the one or more hydrogel polymers further comprise a cell adhesion peptide conjugated to a hydrogel polymer. The cell adhesion peptide can be any of those described hereinabove. In some embodiments, the cell adhesion peptide comprises the amino acid sequence RGD. In some embodiments, the cell adhesion peptide comprises the amino acid sequence CGGNGEPRGDTYRAY (SEQ ID NO: 2).

In some embodiments, the crosslinking agent is a proteolytically cleavable cross-linker peptide. In some embodiments, the peptide is cleavable by a MMP. In some embodiments, the proteolytically cleavable cross-linker peptide comprises the amino acid sequence CQPQGLAKC (SEQ ID NO: 1).

In some embodiments, the lyophilized crosslinked hydrogel matrix is a substantially two-dimensional material, e.g., a sheet or disc. In some embodiments, the sheet or disc has a thickness of between about 1 mm and about 20 mm. In some embodiments, the sheet or disc has a thickness of between about 10 mm and about 20 mm. In some embodiments, the lyophilized crosslinked hydrogel matrix is a powder, e.g., a powder comprising nano- and/or microparticles. In some embodiments, the powder has an average particle size of between about 50 micrometers (μm) and about 500 μm (e.g., about 50, 75, 100, 125, 150, 175, 200, 250, 300, 350, 400, 450, or about 500 μm).

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Hydrogel System

Materials: Hyaluronic acid (HyA, sodium salt, 500 kDa) was purchased from Lifecore Biomedical (Chaska, Minn., United States of America). Adipic dihydrazide (ADH), 1-ethyl-3-[3-(dimethylamino)propyl] carbodiimide (EDC), sodium hydroxide (NaOH), hydrochloric acid (HCl), tris(2-carboxyethyl)phosphine (TCEP), triethanolamine-buffer (TEOA; 0.3 M, pH 8) and 1-hydroxybenzotriazole (HOBt) were purchased from Aldrich (Milwaukee, Wis., United States of America). Dimethyl sulfoxide (DMSO), N-Acryloxysuccinimide (NAS) and ethanol were obtained from Fisher Scientific (Waltham, Mass., United States of America). Dialysis membranes (10000 MWCO, SpectraPor Biotech CE) were purchased from Spectrum Laboratories (Rancho Dominguez, Calif., United States of America). High molecular weight heparin (HMWH) was obtained from Santa Cruz Biotechnology, Inc (Dallas, Tex., United States of America). The MMP-degradable crosslinker peptide (CQPQGLAKC; SEQ ID NO: 1), and bsp-RGD(15) adhesion peptide (CGGNGEPRGDTYRAY; SEQ ID NO: 2) were synthesized by United BioSystem Inc (Herndon, Va., United States of America).

Synthesis of Acrylated HyA: HyA based hydrogels were synthesized according to previously reported methods. See Jha et al., Biomaterials 89, 136-147 (2016); Jha et al., Biomaterials 47, 1-12 (2015); and Jha et al., J. Control. Release 209, 308-316 (2015). Briefly, a HyA derivative carrying hydrazide groups (HyAADH) was synthesized by addition of 30 molar excess of ADH to HyA in deionized (DI) water (100 mL, 3 mg/ml). Solution pH was adjusted to 6.8 using 0.1M NaOH and 0.1M HCl. EDC (3 mmol) and HOBt (3 mmol) were dissolved separately in DMSO/water (1/1 volume ratio, 3 mL) and added to the HyA solution sequentially. The pH was maintained at 6.8 for at least the first 6 h, after which the solution was allowed to react for 24 h. After 24 h, the solution was adjusted to pH 7.0 and exhaustively dialyzed against DI water. N-acryloxysuccinimide (700 mg) was subsequently reacted with the HyAADH solution (300 mg, 100 mL DI water) to generate acrylate groups on the HyA (AcHyA). After 24 h, the product was exhaustively dialyzed against DI water and lyophilized.

Generation of AcHyA-bsp-RGD: The AcHyA-RGD derivative was synthesized by reacting CGGNGEPRGDTYRAY (bsp-RGD(15); SEQ ID NO: 2; see Rezania and Healy, Biotechnol. Prog. 15, 19-32 (1999); and Harbers and Healy, J. Biomed. Mater. Res. Part A 75A, 855-969 (2005)) (10 mg) with AcHyA solution (25 mg, 10 mL DI water) at room temperature. The peptide was pre-treated with excess TCEP in order to reduce any disulfide bonds that had formed between thiol groups. The AcHyA-bsp-RGD product was exhaustively dialysed against DI water, followed by lyophilization.

Synthesis of Thiolated Heparin (heparin-SH): Thiolated-heparin was synthesized according to a previously published method. See Jha et al., Biomaterials 89, 136-147 (2016); Jha et al., Biomaterials 47, 1-12 (2015); and Jha et al., J. Control. Release 209, 308-316 (2015). Briefly, heparin (50 mg, 10 mL DI water) was reacted with an excess of crystamine in the presence of EDC and HOBt at pH 6.8. Next, the reaction solution was exhaustively dialyzed to remove any small molecules not attached to heparin, and then the reaction product was lyophilized. After that, a 10-fold molar excess of TCEP was added to reduce the oxidized disulfide groups. This solution was allowed to react for 3 h at pH 7.5 and then adjusted to pH 5.0 by the addition of 1.0 N HCl. The acidified solution was dialyzed against dilute HCl containing 100 mM NaCl, followed by dialysis against dilute HCl and lyophilization.

AcHyA Hydrogel Formation: Hydrogels were formed as previously described. See Jha et al., Biomaterials 89, 136-147 (2016); and Jha et al., Biomaterials 47, 1-12 (2015). AcHyA (4 mg), AcHyA-RGD (6 mg), and heparin-SH (0.03 wt %) were dissolved in 0.3 mL of TEOA buffer, then HyA hydrogels were fabricated by mixing bis-cysteine containing an MMP cleavable peptide (CQPQGLAKC; SEQ ID NO: 1) (3 mg, 50 μL TEOA buffer) with the solution of HyA precursors.

### Example 2

### Treatment of VML Injury in Tibialis Anterior Model

Experimental Outline: Eleven 12-week old female Lewis rats were surgically given 20% by mass volumetric muscle loss injuries to the left tibialis anterior (TA). The injury was approximately in the center of the side of the TA muscle nearest to the skin surface, leaving some native muscle directly adjacent to the tibia and some native muscle on the side of the TA muscle farthest away from the tibia. Five animals were given the injury with no repair performed (NR, injured) and six were given the injury and immediately treated with an acrylated hyaluronic acid hydrogel (HyA, treated). Maximum isometric torque testing was performed prior to surgery (baseline), and at 4, 8, and 12 weeks post-surgery on all animals. The animals were sacrificed after the 12-week timepoint, and the treated and contralateral TA muscles were explanted for immunohistochemical analysis, where the contralateral TA muscles were used as comparative controls.

A total of 24 female Lewis rats (Charles River Laboratories, Wilmington, Mass., United States of America) weighing 180.0±7.7 g at 12 weeks of age were pair housed in a vivarium and were provided with food and water ad libitum.

Creation of VML Injury: VML injuries were surgically created as previously described. See Wu et al., Biores. Open Access 1, 280-290 (2012); and Corona et al., Tissue Eng. Part A 131219054609007 (2013). Briefly, a longitudinal incision was made on the lateral portion of the lower left leg. The skin was then cleared from the underlying fascia using blunt separation, and the fascia covering the anterior crural muscles was separated using blunt dissection. The proximal and distal tendons of the Extensor Hallicus Longus (EHL) and Extensor Digitorum Longus (EDL) muscles were then isolated and ablated. As previously described, the TA muscle corresponds to 0.17% of the gross body weight. See Wu et al., Biores. Open Access 1, 280-290 (2012); and Corona et al., Tissue Eng. Part A 131219054609007 (2013). The VML injury model was characterized by excision of about 20% of the TA muscle weight from the middle third of the muscle. The fascia was closed with 6-0 vicryl sutures and the skin was closed with 5-0 prolene using interrupted sutures. Skin glue was applied over the skin sutures to help prevent the incision from opening. For HyA-heparin treated animals, the hydrogel was injected immediately following closure of the fascia. Once the injection was complete, skin closure continued as normal. The animal remained sedated for 30 minutes to allow the gel to crosslink.

Force Testing: In vivo functional testing was performed as previously described. See Mintz et al., J Vis Exp. (2016). J Vis Exp. 116, e54487 (2016). Briefly, at 4, 8 and 12 weeks relative to the surgery date, rats were anesthetized, and the left hind limb was aseptically prepared. The rat was placed in a supine position on a heated platform and the left knee was bent to a 90° angle. The leg was secured using a stabilizing rod and the left foot was taped to a footplate. The footplate was attached to the shaft of an Aurora Scientific 305C-LR-FP servomotor (Aurora Scientific Inc., Aurora, Ontario, Canada), which was controlled using a computer. Sterilized percutaneous needle electrodes were carefully inserted into the skin of the lower left leg for stimulation of the left common peroneal nerve. Electrical stimulus was provided using a stimulator with a constant current SIU (Model 701C; Aurora Scientific Inc., Aurora Ontario, Canada). Stimulation voltage and needle electrode placement were optimized with a series of 1 Hz pulses resulting in twitch contraction. Contractile function of the anterior crural muscles was assessed through measuring the peak isometric tetanic torque determined from maximal response to a series of stimulation frequencies (10-150 Hz). Torque at baseline was normalized by the body weight of each animal. Torque at each post-surgical timepoint was normalized by the body weight of each animal on the day of collection, then was normalized to a percent of the baseline for that animal. The normalized torques at each post-surgical timepoint were averaged for analysis. After functional testing, the animals were allowed to recover on the heated platform and were then returned to the vivarium. For terminal time points, animals were euthanized via CO₂ inhalation and cervical dislocation was performed as a secondary measure.

Histology and Immunohistochemistry: All samples were fixed in 4% paraformaldehyde, then processed and embedded in paraffin. Serial transverse sections (7 μm) were cut from the paraffin embedded blocks and stained with hematoxylin and eosin (H&E). Cross-sectional areas of ≈200 muscle fibers in the outer and inner portions of TA muscle were measured using ImageJ software (National Institutes of Health, Bethesda, Md., United States of America).

Immunohistochemical staining was performed using rabbit anti-CD31/PECAM1 antibody (NB100-2284, Novus Biological, Centennial, Colo., United States of America) and stained with a biotinylated goat anti-rabbit IgG (BA-1000, Vector Laboratories Inc.; Burlingame, Calif., United States of America). The sections were next treated with Avidin Biotin Complex Reagent (PK-7100, Vector Laboratories Inc., Burlingame, Calif., United States of America) and visualized using a NovaRED substrate kit (SK-4800, Vector Laboratories Inc., Burlingame, Calif., United States of America). Tissue sections without primary antibody were used as negative controls. Images were captured and digitized (DM4000B Leica Upright Microscope, Leica Microsystems, Wetlzar, Germany). Capillaries were quantified by counting the number of CD31+ cells around individual fibers (at least 100 fibers counted per sample).

Statistics: Numerical data are presented as mean±standard error of the mean (SEM). Morphological and functional data between the three groups (control, NR, HyA) were analyzed using one- and two-way analyses of variance (ANOVA) or T test as indicated in the figure captions. Upon finding a significant ANOVA, post-hoc comparison testing of parameters of interest was performed using Sidak's least significant difference (LSD) test at α-level 0.05. The statistical significance of the fiber cross-sectional area (FCSA) frequency distribution was determined using Krushal-Wallis with Dunn's multiple comparisons test. Statistical analyses were conducted using GraphPad Prism 7.0 (La Jolla, Calif., United States of America).

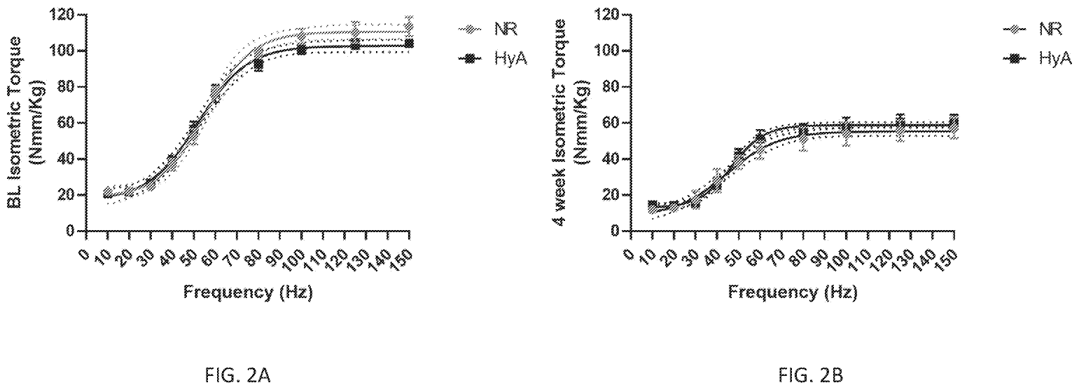

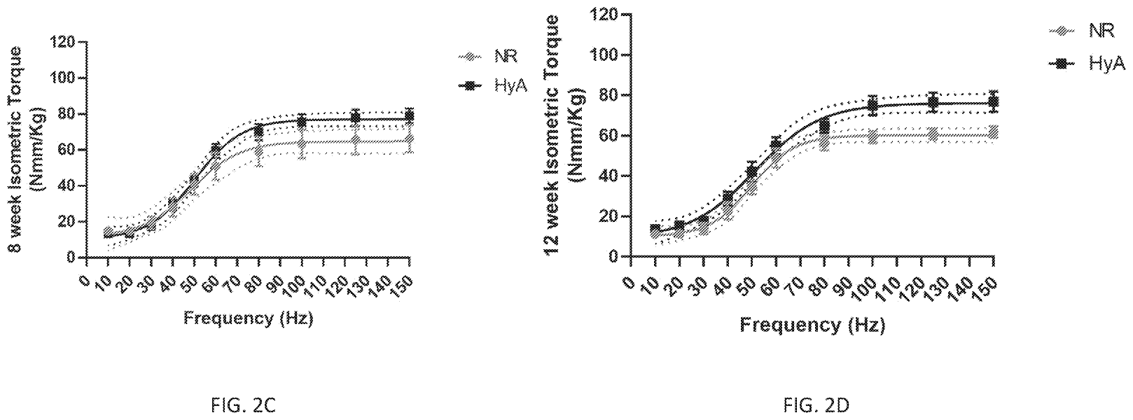

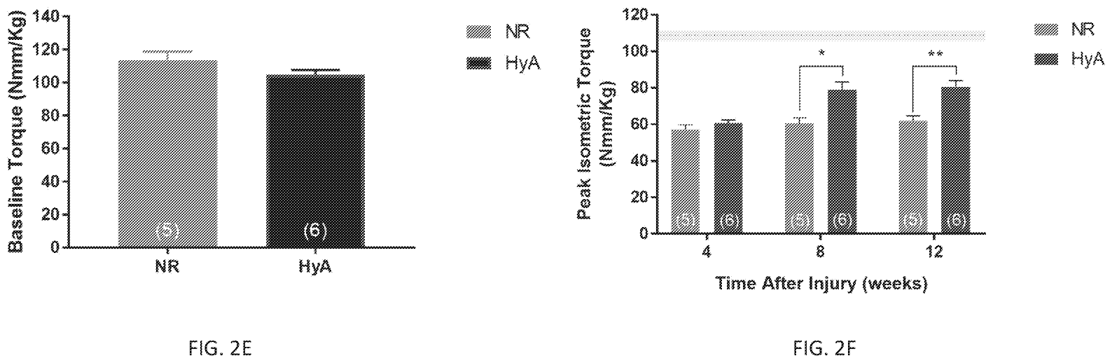

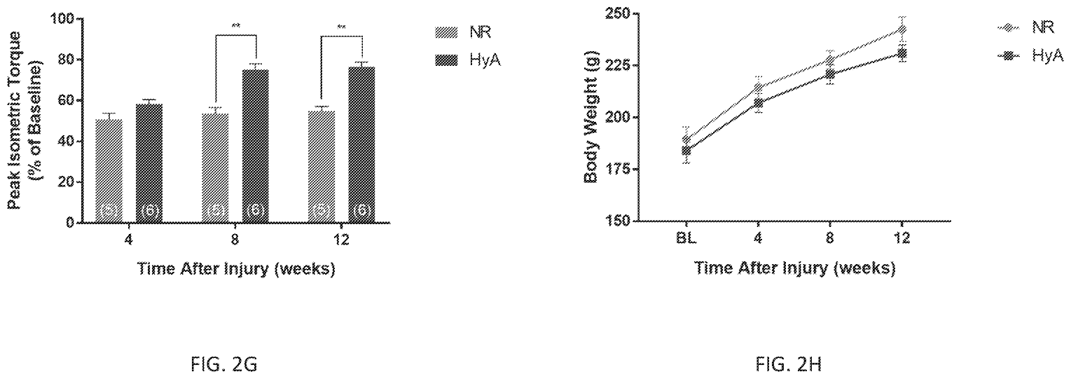

Discussion: None of the animals in the study died during the surgical procedure, no post-implantation mortality occurred, and animals exhibited normal healthy weight gain in all treatment groups over the course of 12 weeks. There was no significant difference between the mean animal body weights (Two-Way ANOVA, p=0.05, FIG. 2H) between the groups over the course of the study or the torque generated by the HA and NR groups at baseline (Unpaired t-Test, p=0.05, FIG. 2E) between the groups over the entire course of the study. However, because animals gained weight over the course of the study, all statistical comparisons on functional measures were made on data normalized to body weight. Post-surgical isometric tetanic dorsiflexion torque testing at 8 and 12 weeks showed a significant increase in the torque generated by the HA group compared to the NR group (Two-Way ANOVA followed by Sidak's post-hoc test, p<0.05). Isometric torque-frequency response at baseline, 4-, 8- and 12-weeks post injury is shown in FIGS. 2A-2D. The average maximum torques as a percentage of baseline measured for the HA and NR groups were 55.9±6.1% and 48.7±6.9% at 4 weeks, 71.9±8.3% and 52.2±7.0% at 8 weeks, and 73.4±5.8% and 52.63±6.07% at 12 weeks. See FIGS. 2F-2G.

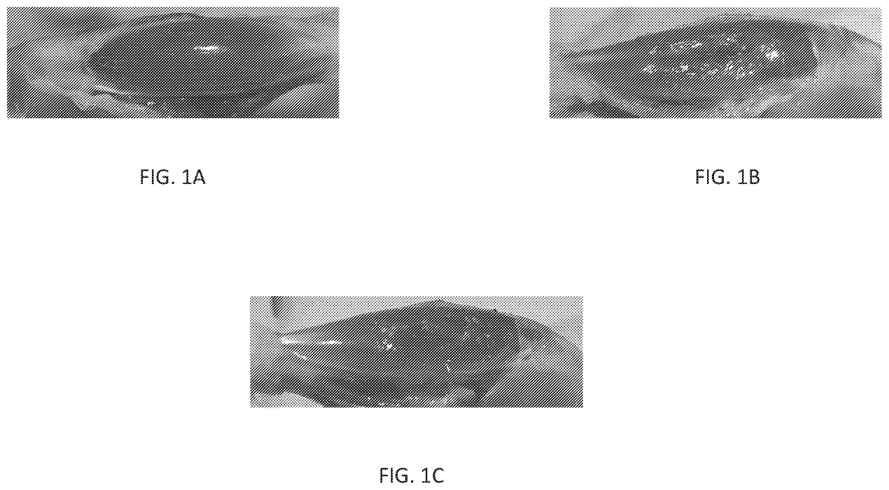

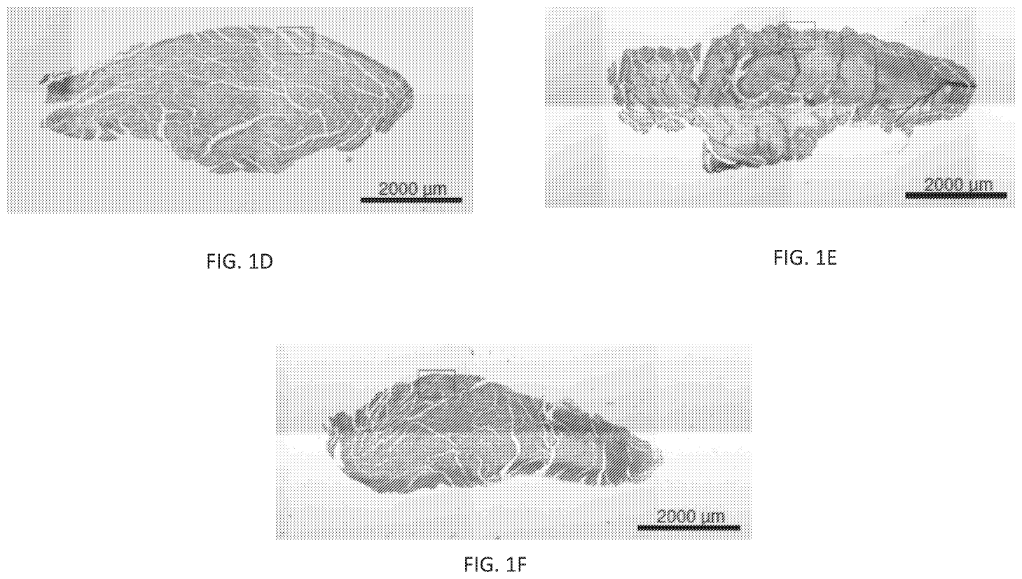

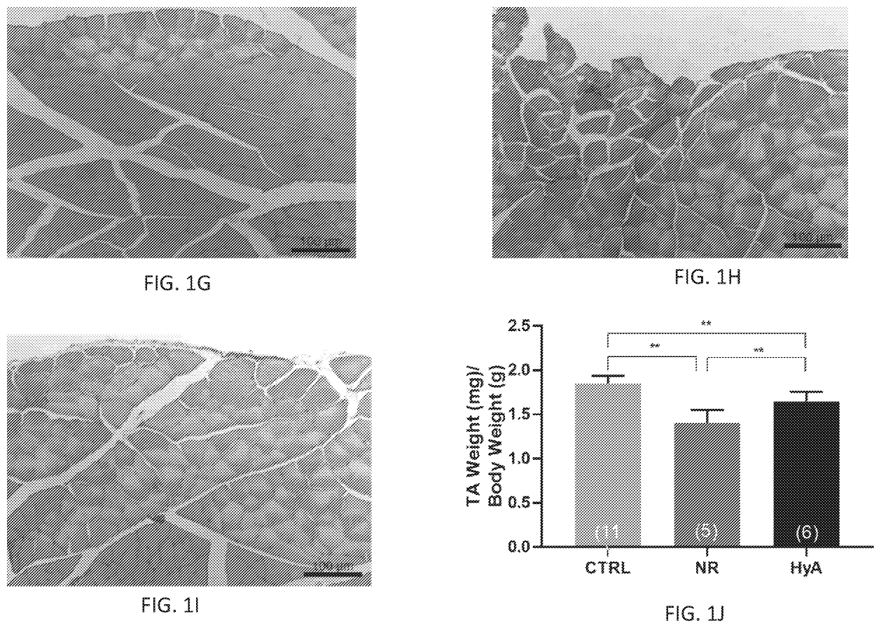

Macroscopically, the hydrogel injection was well tolerated by the recipient animals, with no signs of infection, seroma, or rejection. Significant remodeling of the defect site was evident 12 weeks after creation of the VML injury. See FIGS. 1A-1C. Representative histology from tissue sections in both groups are shown in FIGS. 1E, 1F, 1H, and 1I. These images were obtained in the first 400 μm from the surface of the TA, in the center of the muscle belly where the defect was originally created. Representative histology from tissue sections of non-injured muscle are shown in FIGS. 1D and 1G.

At 12 weeks post-injury, HyA-treated animals showed significant restoration of tissue morphology as compared to the NR group. See FIGS. 1A-1C. The TA weight/body weight of NR and HyA-treated animals was significantly lower than control. However, TA muscles treated with HyA presented a statistically significant gain in mass compared to the NR group, indicating regenerative effect from the HyA treatment. See FIG. 1J. The average mass of the explanted treated TA muscles from the HyA animals was 10.9±5.6% lower than the contralateral control, but 17.5±0.4% higher than the explanted injured TA muscle of the NR animals. Importantly, there was little evidence of tissue adhesions and/or fibrotic scarring near the wound site, as typically observed even in treated animals with physiologically significant functional recovery.

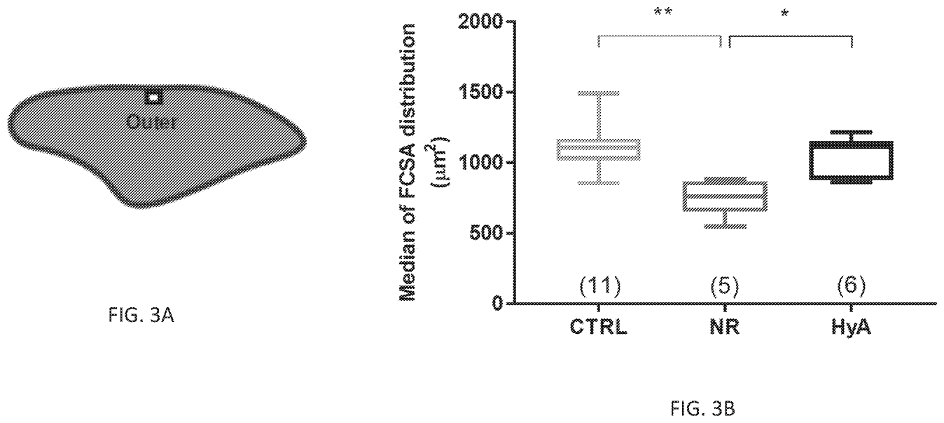

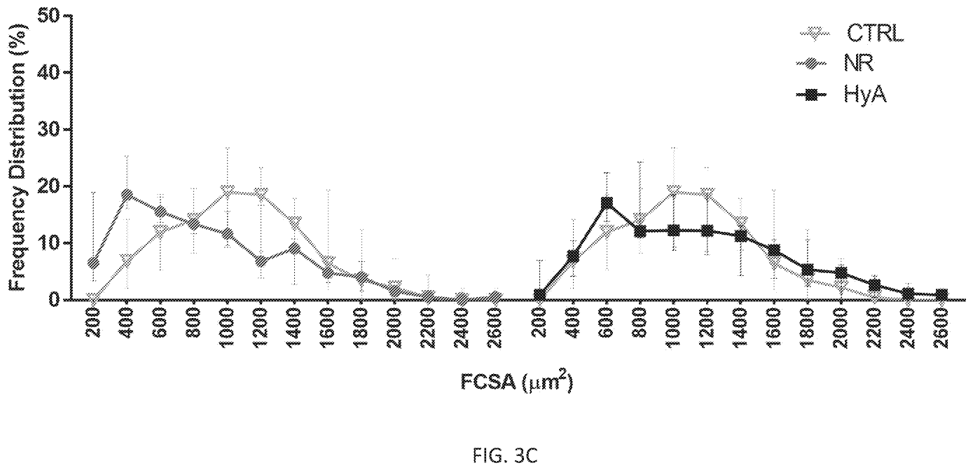

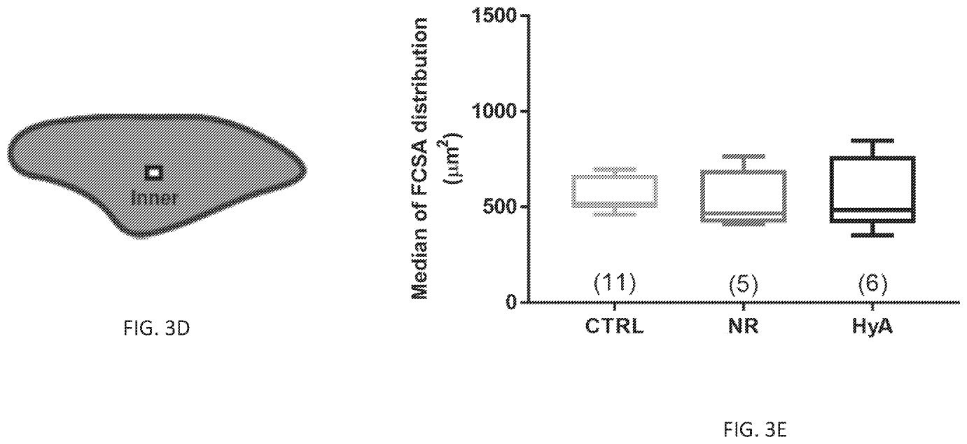

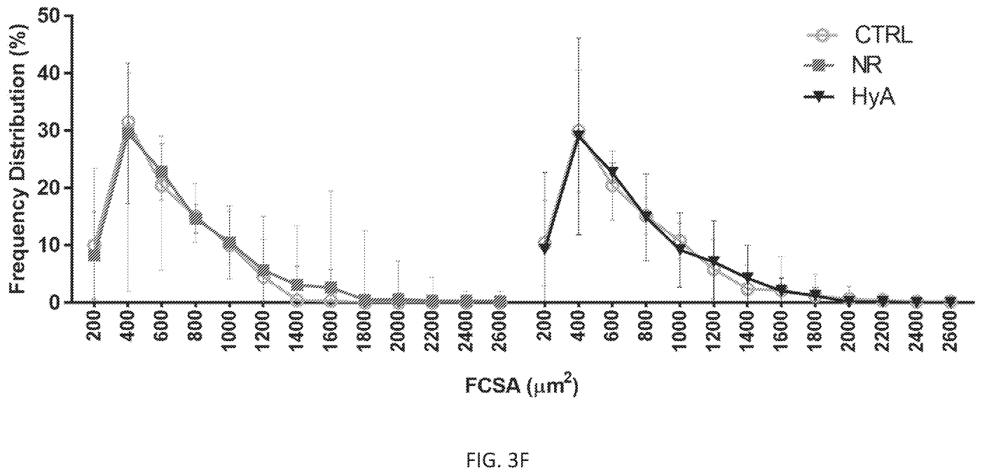

In both groups, the fiber cross-sectional area (FCSA) in the outer region of the TA (see FIG. 3A) presented as a non-normal distribution (D'Agostino & Pearson normality test, p>0.05). See FIG. 3C. The median of the FCSA distribution in the TA of the NR animals (762±131.5 μm²) was significantly smaller than both the HyA animals (1055±145.1 μm², p<0.05) and the contralateral control muscles (1112±167.8 μm², p<0.05). See FIG. 3B. No statistical difference was observed between control and hydrogel-treated TAs. In the inner region of the TA muscle (see FIG. 3D) there was no significant difference among the median FCSA when comparing the TAs of the HyA animals, nonrepaired (NR) animals and the contralateral controls (One-Way ANOVA with Tukey's multiple comparisons test, p>0.05), suggesting no deleterious effects outside the surgically injured area. See FIGS. 3E and 3F.

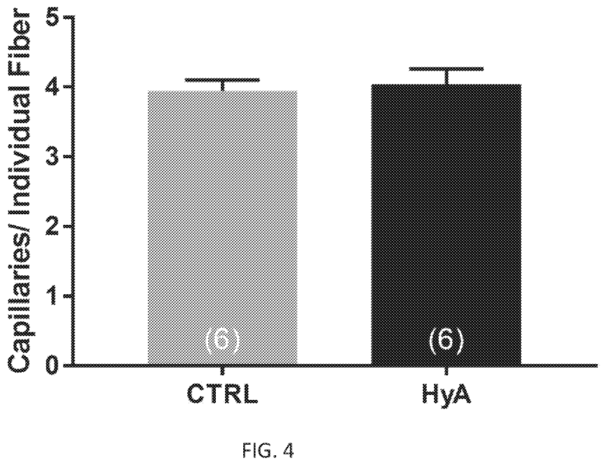

Vascularization is a critical component of normal skeletal muscle function, and an absolute prerequisite for functional regeneration. As such, revascularization of the HyA-implanted defect region was evaluated. The number of CD31+ cells surrounding fibers in the region of the HyA treated group that nominally was the site of de novo muscle fiber regeneration were quantified. No statistical differences were found between the HyA-treated group and the control muscles (4.0±0.6 vs. 3.9±0.4, p>0.05 after T-test). See FIG. 4.

Summary: If one uses statistically significant increases in maximal isometric torque compared to the NR group as an indication of functional recovery, then one concludes that the implantation of HyA-heparin hydrogel as disclosed herein has a positive impact. See FIGS. 2F and 2G. To put these observations into context, it should be noted that the TA synergist muscles (EDL and EHL) were ablated at the time of creation of the surgical TA VML injury, resulting in a permanent approximate 20% functional deficit. See Corona et al., Tissue engineering. Part A 20, 705-715 (2014). This means that after removal of 20-30% of the TA muscle, the total functional deficit is approximately 50%, of which only 30% is recoverable. Therefore, the maximal functional recovery possible is 80% of the preinjury baseline maximal isometric torque response. The NR group exhibited a mean maximal torque response of 52.6%, whereas the HyA group exhibited mean maximal values of 71.9% and 73.4% at 8 and 12 weeks, respectively. These values represent robust functional recoveries of 89.8% and 91.7% when compared to the 80% maximum. This represents the first instance of significant recovery of function observed at 8 weeks post-implantation in this animal model of VML injury, illustrating a substantial leftward shift in the recovery timeline. Without being bound to any one theory or mechanism of action, the other data (see e.g., FIG. 4) support the supposition that this shift is related to the enhanced angiogenic potential of the HyA-based matrix and migration of muscle satellite cells into the matrix during wound healing.

Qualitative and quantitative morphological and histological analyses also provide insights into the potential mechanisms responsible for the observed functional recovery in the HyA group. For example, consistent with the robust functional recovery observed, as illustrated by the representative examples and data in FIGS. 1A-1J and FIGS. 3B, 3C, 3E, and 3F, the TA muscles treated with HyA-heparin hydrogels showed significant volume reconstitution. More particularly, the HyA-treated animals showed significantly greater mass than the NR animals, as well as decreased fibrotic infiltration compared to the NR animals in this study. Since no donor cells were added, and moreover, as there was no evidence for fibrosis well within the implanted region, it is concluded that the volume/mass recovery was related to de novo muscle fiber regeneration. The fact that there was no significant difference in FCSA or the capillary density between the HyA group and the contralateral control muscles, also supports the conclusion that the HyA-heparin hydrogel treatment can effectively contribute to significant muscle regeneration and revascularization following implantation in a VML injury defect site.

More particularly, HyA-heparin hydrogel implantation was associated with significantly increased new tissue formation, representing relatively mature native-like fibers as compared to the NR group. In fact, smaller diameter fibers with centrally located nuclei were easily identified in regions of surgical excision in the NR animals (i.e. the wound bed and empty space between the two sides of the TA muscle belly). These are established indicative markers for new muscle tissue formation (see Aurora et al., BMC Sport. Sci. Med. Rehabil. 6, 41 (2014); Corona et al., Am. J. Physiol. Physiol. 305, C761-C775 (2013); and Hawke and Garry, J. Appl. Physiol. 91, 534-551 (2001), and they are consistent with an ongoing repair/remodeling process that was particularly apparent in the NR group, and, without being bound to any one theory, likely reflects a cycle of damage and repair due to the 30% decrease in muscle mass (e.g., overload injury), as well as excision of the synergist muscles. See Corona et al., Biomaterials 34, 3324-3335 (2013). These observations are consistent with the supposition that the HyA-heparin hydrogel treatment promotes a microenvironment that is more favorable for muscle regeneration and remodeling, resulting in an apparent “active zone of regeneration” spanning from the border of the native tissue to regions well within the VML excision area. As shown in FIGS. 1A-1J, 3B, 3C, and 4, there was nearly a complete restoration of tissue volume and structure. This observation was supported by measurement of the average masses of the explanted muscles from each group, most notably by the significant increase in volume reconstitution in the treated group as compared to the NR animals.

In summary, as is believed to be shown herein for the first time, heparin-conjugated hyaluronic acid hydrogels can successfully promote a significant functional recovery in instances of severe skeletal muscle damage in an established and biologically relevant rodent model of TA VML injury. HyA-heparin hydrogel implantation resulted in significant functional recovery at 8- and 12-weeks post-injury and the resulting recovery of muscle structure and volume is due to de novo muscle regeneration, resulting in muscle tissue that was nearly indistinguishable from native muscle. Taken together, these observations have important implications for regenerative therapeutics for VML injuries and VML-like conditions, as the hydrogel is a highly tunable biomaterial and growth factor sequestration platform that could vastly extend the potential range of clinical applications.

### Example 3

### Treatment of VML Injury in Latissimus Dorsi Model

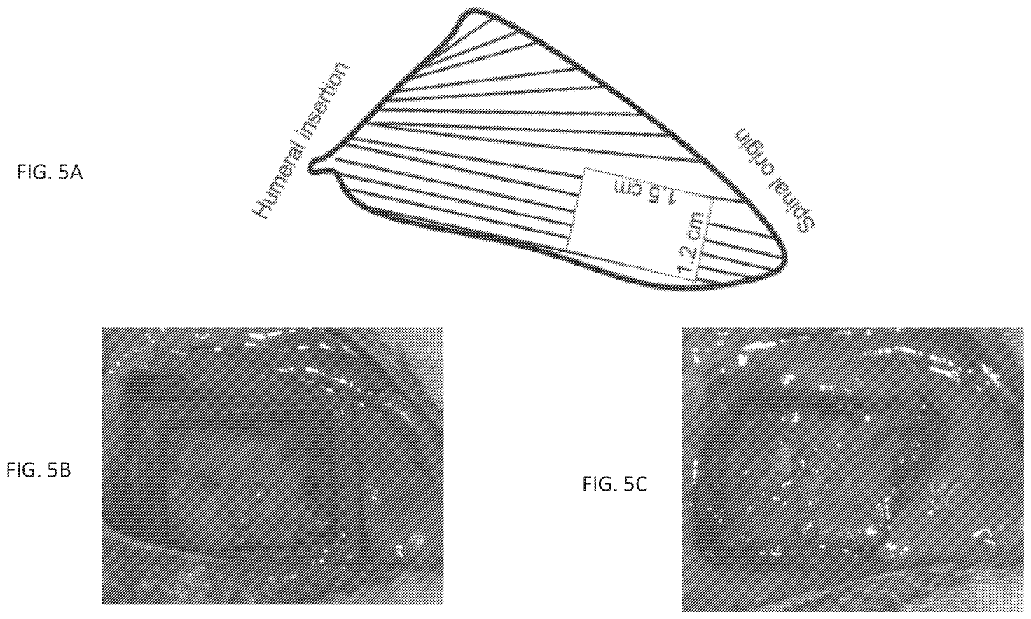

A VML injury was surgically created using a previously described LD model. See Passipieri et al., Tissue Eng. Part A, published online Mar. 18, 2019 (doi.org/10.1089/ten.tea.2018.0280). FIG. 5A provides a schematic representation of the location and size of the surgical injury in the latissimus dorsi (LD) muscle to mimic VML in the rat. The LD muscle is a pennate muscle. Injury is created near the spinal origin, and it removes a portion of the fibers that are oriented parallelly. Repair of the LD was performed by inserting a 3D-printed square mold over the surgically created muscle defect and then applying solutions of the hydrogel precursors into the space within the mold. See FIG. 5B. The mold helps the hydrogel stay in a desired location before it solidifies via crosslinking of the hydrogel precursors. Solidification occurred within 10 minutes, after which the mold was removed, leaving the solidified HyA hydrogel in place inside the muscle defect. See FIG. 5C. While LD muscles retrieved one hour after HyA implantation showed that the hydrogel could stay secured to the muscle defect area, the consistency of the hydrogel also allowed placement of sutures, if desired, further securing the hydrogel to the muscle.

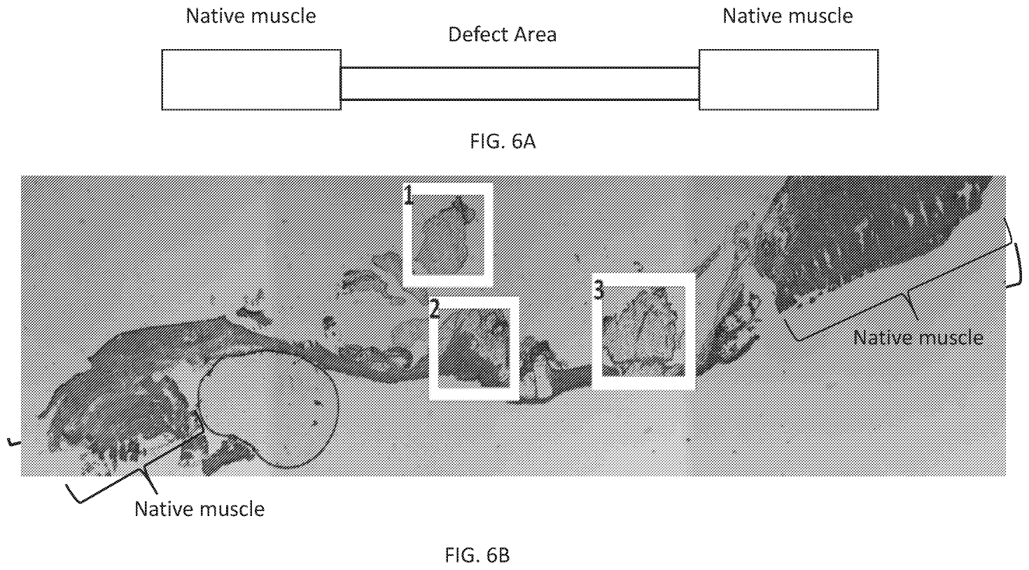

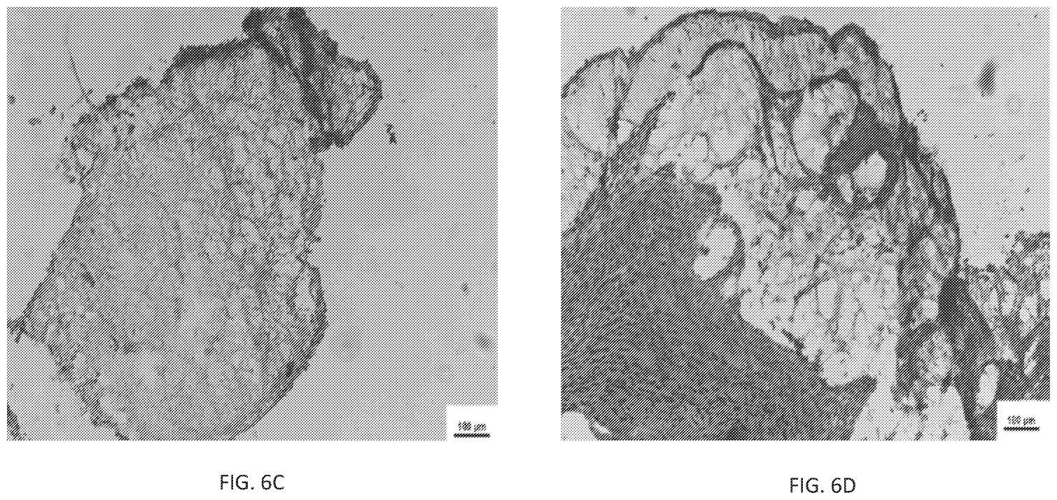

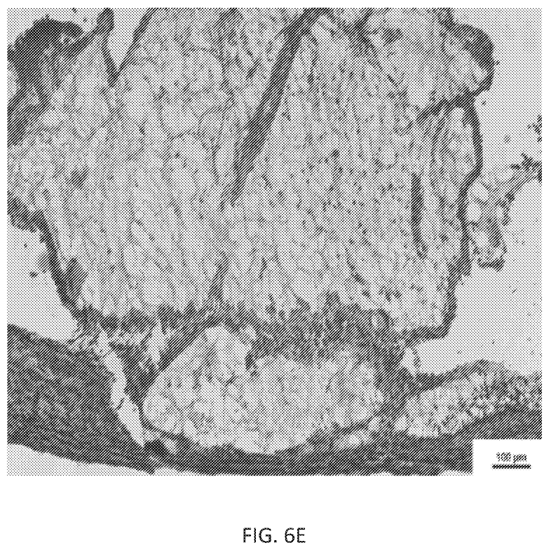

FIG. 6B shows the H&E staining image of a cross-sectional cut of a hydrogel treated LD muscle retrieved five days after implantation of the HyA hydrogel. As illustrated schematically in FIG. 6A, the middle of the cross-section corresponds to the defect/HyA-treated site, while the sides correspond to native muscle. Hydrogel was still present in the defect area. See FIGS. 6B and 6C. As shown in FIG. 6B and the higher magnification images of FIGS. 6D and 6E, cell infiltration was observed in the hydrogel.

### Example 4

### Hydrogel Swelling

The Hya-hydrogel of Example 1 was frozen at −80° C. and lyophilized at −88° C. and less than 0.05 mBar for 2 days. Discs (approximately 4 mg) were cut out for swelling studies.

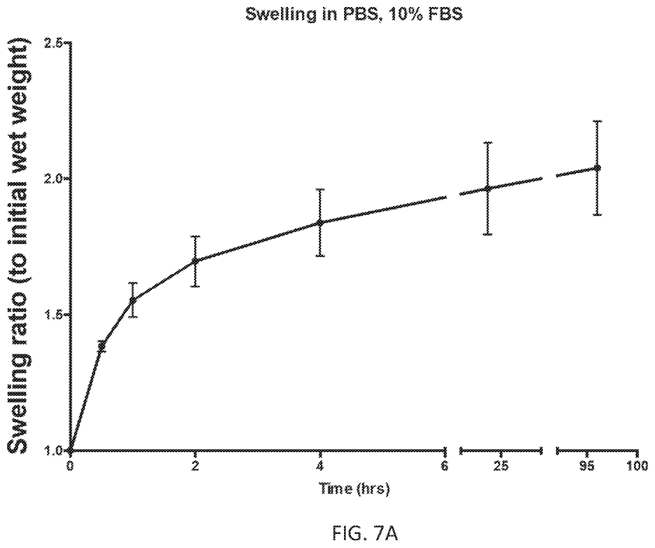

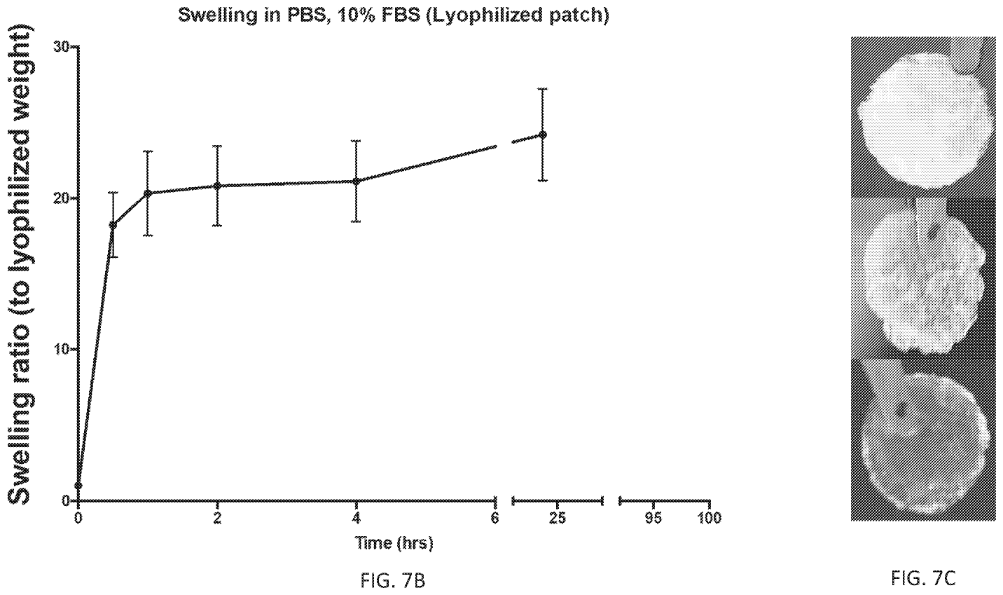

The swelling of a 500 kDa HyA hydrogel and of a 4 mg disc cut from a sheet of the freeze-dried 500 kDa HyA hydrogel were studied in 1 milliliter phosphate buffered saline (PBS) containing 10% by volume fetal bovine serum (FBS) at 37° C. (to mimic swelling conditions in vivo). At various time points the hydrogel samples were removed from the PBS solution and weighed to determine swelling. Results are shown in FIGS. 7A and 7B. The freeze-dried sample of the hydrogel was able to swell to 20 times its original dry weight in less than 2 hours, indicating that the freeze-dried hydrogel could be used as a convenient, easily transportable form of the hydrogel which could be cut into any desirable shape as needed to fill a particular injury and would swell after administration to fill a muscle injury site quickly. FIG. 7C shows how the white opaque freeze-dried hydrogel circle gradually becomes transparent as it swells. The non-freezedried sample swelled to more than 1.5 times its original weight in two hours and about 2 times its original weight over the course of about 4 days.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of treating volumetric muscle loss (VML) injury, the method comprising:
(a) providing a subject in need of VML treatment; and
(b) administering to the subject a regenerative hydrogel system, wherein said regenerative hydrogel system comprises a growth factor recruitment moiety, optionally heparin or a derivative or copolymer thereof, and wherein the regenerative hydrogel system is free of exogenous growth factors and biological cells,
whereby the VML in the subject is treated, optionally wherein the treating provides between about 20 percent (%) and about 99% muscle function recovery, further optionally wherein the treating provides between about 50% and about 99% muscle function recovery.

2. The method of claim 1, wherein the regenerative hydrogel system comprises a plurality of hydrogel matrix precursors, wherein said precursors comprise: (i) one or more hydrogel polymers; (ii) a growth factor recruitment moiety, optionally heparin or a derivative thereof, further optionally wherein the heparin is conjugated to one or more hydrogel polymers; and (iii) a proteolytically cleavable cross-linker peptide; and wherein the administering of step (b) comprises administering the plurality of hydrogel matrix precursors to a muscle injury site in the subject, whereby the precursors form a crosslinked hydrogel matrix in situ in the muscle injury site, wherein said crosslinked hydrogel matrix comprises one or more hydrogel polymers, wherein said one or more hydrogel polymers comprises a growth factor recruitment moiety-conjugated hydrogel polymer, and wherein the proteolytically cleaveable cross-linker agent links one of the one or more hydrogel polymers to another of the one or more hydrogel polymers.

3. The method of claim 2, wherein the administering comprises placing a removable mold in the muscle injury site prior to the administering of step (b) to define an area where the crosslinked hydrogel matrix is to be formed and where the plurality of hydrogel precursors are to be administered; and wherein the method further comprises removing the mold from the injury site following the administration of step (b), optionally wherein the mold is removed about 10 minutes after the administering of step (b).

4. The method of claim 2 or claim 3, wherein the method further comprises suturing the crosslinked hydrogel matrix in place in the muscle injury site.

5. The method of claim 1, wherein the regenerative hydrogel system comprises a lyophilized crosslinked hydrogel matrix material comprising: (i) one or more hydrogel polymers, (ii) a growth factor recruitment moiety, optionally heparin, conjugated to one or more hydrogel polymer; and (iii) a proteolytically cleavable cross-linker peptide, wherein the proteolytically cleavable cross-linker peptide links one of the one or more hydrogel polymers to another of the one or more hydrogel polymers.

6. The method of claim 5, wherein the lyophilized crosslinked hydrogel matrix material is administered to a muscle injury site in the subject as a sheet or disc comprising the lyophilized crosslinked hydrogel matrix material.

7. The method of claim 6, wherein the method further comprises suturing the sheet or disc in place in the muscle injury site.

8. The method of claim 5, wherein the lyophilized crosslinked hydrogel matrix material is administered to a muscle injury site in the subject in powder form.

9. The method of any one of claims 2-8, wherein the one or more hydrogel polymers comprise an acrylated hyaluronic acid polymer (HyA).

10. The method of any one of claims 2-9, wherein the one or more hydrogel polymers further comprise a hydrogel polymer conjugated to a cell adhesion peptide.

11. The method of claim 10, wherein the cell adhesion peptide comprises the amino acid sequence RGD.

12. The method of claim 11, wherein the cell adhesion peptide comprises the amino acid sequence CGGNGEPRGDTYRAY (SEQ ID NO: 2).

13. The method of any one of claims 2-12, wherein the proteolytically cleavable cross-linker peptide comprises the amino acid sequence CQPQGLAKC (SEQ ID NO: 1).

14. The method of any one of claims 1-13, wherein the growth factor recruitment moiety comprises heparin, optionally a high molecular weight heparin (HMWH) or a derivative or copolymer thereof, further optionally wherein the HMWH has a weight average molecular weight (MW_{w}) of between about 6 kilodaltons (kDa) and about 12 kDa.

15. The method of any one of claims 1-14, wherein administration of the regenerative hydrogel system provides improved muscle recovery compared to a non-treated muscle injury, optionally wherein said improved muscle recovery comprises one or more of increased muscle mass compared to a non-treated muscle injury, increased muscle volume compared to a non-treated muscle injury, improved muscle vascularization compared to a non-treated injury, or improved muscle function compared to a non-treated muscle injury.

16. The method of any one of claims 1-15, wherein administration of the regenerative hydrogel system provides increased muscle mass and/or increased muscle function compared to a non-treated muscle injury within eight to twelve weeks after administration.

17. The method of any one of claims 1-16, wherein the subject is a human.

18. A lyophilized crosslinked hydrogel matrix comprising (i) one or more hydrogel polymers, (ii) a growth factor recruitment moiety, optionally heparin, conjugated to one or more hydrogel polymer; and (iii) a proteolytically cleavable cross-linker peptide, wherein the proteolytically cleavable cross-linker peptide links one of the one or more hydrogel polymers to another of the one or more hydrogel polymers.

19. The lyophilized crosslinked hydrogel matrix of claim 18, wherein the one or more hydrogel polymers comprises an acrylated hyaluronic acid polymer (HyA).

20. The lyophilized crosslinked hydrogel matrix of claim 18 or claim 19, wherein the growth factor recruitment moiety comprises heparin, optionally a high molecular weight heparin (HMWH), further optionally wherein the HMWH has a weight average molecular weight (MW_{w}) of between about 6 kilodaltons (kDa) and about 12 kDa.

21. The lyophilized crosslinked hydrogel matrix of any one of claims 18-20, wherein the one or more hydrogel polymers further comprises a cell adhesion peptide conjugated to a hydrogel polymer.

22. The lyophilized crosslinked hydrogel matrix of claim 21, wherein the cell adhesion peptide comprises the amino acid sequence RGD.

23. The lyophilized crosslinked hydrogel matrix of claim 22, wherein the cell adhesion peptide comprises the amino acid sequence CGGNGEPRGDTYRAY (SEQ ID NO: 2).

24. The lyophilized crosslinked hydrogel matrix of any one of claims 18-23, wherein the proteolytically cleavable cross-linker peptide comprises the amino acid sequence CQPQGLAKC (SEQ ID NO: 1).

25. A sheet or disc comprising the lyophilized crosslinked hydrogel matrix of any one of claims 18-24.

26. The sheet or disc of claim 25, wherein the sheet or disc has a thickness of between about 1 millimeter (mm) and about 10 mm.

27. A powder comprising particles of the lyophilized crosslinked hydrogel matrix of any one of claims 18-24, optionally wherein said powder has an average particle size of between about 50 micrometers (μm) and about 500 μm.


SEQUENCE LISTING (48 sequences)

SEQ ID NO: 1 - protein, 9 aa, Artificial Sequence
  synthetic polypeptide
CQPQGLAKC

SEQ ID NO: 2 - protein, 15 aa, Artificial Sequence
  synthetic polypeptide
CGGNGEPRGDTYRAY

SEQ ID NO: 3 - protein, 7 aa, Artificial Sequence
  synthetic polypeptide
FHRRIKA

SEQ ID NO: 4 - protein, 15 aa, Artificial Sequence
  synthetic polypeptide
  MOD_RES (1)..(1): ACETYLATION
  MOD_RES (15)..(15): AMIDATION
CGGNGEPRGDTYRAY

SEQ ID NO: 5 - protein, 10 aa, Artificial Sequence
  synthetic polypeptide
  MOD_RES (1)..(1): ACETYLATION
  MOD_RES (10)..(10): AMIDATION
CGGFHRRIKA

SEQ ID NO: 6 - protein, 12 aa, Artificial Sequence
  synthetic polypeptide
CEPRGDTYRAYG

SEQ ID NO: 7 - protein, 12 aa, Artificial Sequence
  synthetic polypeptide
CGGGEAPRGDVY

SEQ ID NO: 8 - protein, 10 aa, Artificial Sequence
  synthetic polypeptide
CCGPRGDVYG

SEQ ID NO: 9 - protein, 18 aa, Artificial Sequence
  synthetic polypeptide
CGGVSWFSRHRYSPFAVS

SEQ ID NO: 10 - protein, 15 aa, Artificial Sequence
  synthetic polypeptide
CGGNRWHSIYITRFG

SEQ ID NO: 11 - protein, 15 aa, Artificial Sequence
  synthetic polypeptide
CGGTWYKIAFGRNRK

SEQ ID NO: 12 - protein, 15 aa, Artificial Sequence
  synthetic polypeptide
CGGRKRLQVQLSIRT

SEQ ID NO: 13 - protein, 15 aa, Artificial Sequence
  synthetic polypeptide
CGGKAFDITYVRLKF

SEQ ID NO: 14 - protein, 10 aa, Artificial Sequence
  synthetic polypeptide
CTRKKHDNAQ

SEQ ID NO: 15 - protein, 15 aa, Artificial Sequence
  synthetic polypeptide
VSWFSRHRYSPFAVS

SEQ ID NO: 16 - protein, 10 aa, Artificial Sequence
  synthetic polypeptide
RNIAEIIKDI

SEQ ID NO: 17 - protein, 12 aa, Artificial Sequence
  synthetic polypeptide
TAGSCLRKFSTM

SEQ ID NO: 18 - protein, 10 aa, Artificial Sequence
  synthetic polypeptide
TTSWSQCSKS

SEQ ID NO: 19 - protein, 13 aa, Artificial Sequence
  synthetic polypeptide
RYVVLPRPVCFEK

SEQ ID NO: 20 - protein, 5 aa, Artificial Sequence
  synthetic polypeptide
EVLLI

SEQ ID NO: 21 - protein, 4 aa, Artificial Sequence
  synthetic polypeptide
  MISC_FEATURE (2)..(3): the amino acid in this position can be any amino acid
  MISC_FEATURE (4)..(4): the amino acid in this position can be any hydrophobic amino acid
PXXX

SEQ ID NO: 22 - protein, 5 aa, Artificial Sequence
  synthetic polypeptide
  MISC_FEATURE (2)..(3): the amino acid in this position can be any amino acid
  MISC_FEATURE (4)..(4): the amino acid in this position can be any hydrophobic amino acid
  MISC_FEATURE (5)..(5): the amino acid in this position can be either Ser or Thr
PXXXX

SEQ ID NO: 23 - protein, 6 aa, Artificial Sequence
  synthetic polypeptide
  MISC_FEATURE (2)..(2): the amino acid in this position can be either Leu or Gln
PXGMTS

SEQ ID NO: 24 - protein, 5 aa, Artificial Sequence
  synthetic polypeptide
  MISC_FEATURE (2)..(2): the amino acid in this position can be either Leu or Gln
PXGMT

SEQ ID NO: 25 - protein, 10 aa, Artificial Sequence
  synthetic polypeptide
CGLVPAGSGP

SEQ ID NO: 26 - protein, 12 aa, Artificial Sequence
  synthetic polypeptide
SLLKSRMVPNFN

SEQ ID NO: 27 - protein, 12 aa, Artificial Sequence
  synthetic polypeptide
SLLIARRMPNFN

SEQ ID NO: 28 - protein, 12 aa, Artificial Sequence
  synthetic polypeptide
SKLVQASASGVN

SEQ ID NO: 29 - protein, 12 aa, Artificial Sequence
  synthetic polypeptide
SSYLKASDAPDN

SEQ ID NO: 30 - protein, 12 aa, Artificial Sequence
  synthetic polypeptide
RPKPQQFFGLMN

SEQ ID NO: 31 - protein, 12 aa, Artificial Sequence
  synthetic polypeptide
SLRPLALWRSFN

SEQ ID NO: 32 - protein, 12 aa, Artificial Sequence
  synthetic polypeptide
SPQGIAGQRNFN

SEQ ID NO: 33 - protein, 14 aa, Artificial Sequence
  synthetic polypeptide
DVDERDVRGFASFL

SEQ ID NO: 34 - protein, 12 aa, Artificial Sequence
  synthetic polypeptide
SLPLGLWAPNFN

SEQ ID NO: 35 - protein, 12 aa, Artificial Sequence
  synthetic polypeptide
SLLIFRSWANFN

SEQ ID NO: 36 - protein, 12 aa, Artificial Sequence
  synthetic polypeptide
SGVVIATVIVIT

SEQ ID NO: 37 - protein, 12 aa, Artificial Sequence
  synthetic polypeptide
SLGPQGIWGQFN

SEQ ID NO: 38 - protein, 12 aa, Artificial Sequence
  synthetic polypeptide
KKSPGRVVGGSV

SEQ ID NO: 39 - protein, 12 aa, Artificial Sequence
  synthetic polypeptide
PQGLLGAPGILG

SEQ ID NO: 40 - protein, 31 aa, Artificial Sequence
  synthetic polypeptide
HGPEGLRVGFYESDVMGRGHARLVHVEEPHT

SEQ ID NO: 41 - protein, 12 aa, Artificial Sequence
  synthetic polypeptide
GPQGLAGQRGIV

SEQ ID NO: 42 - protein, 12 aa, Artificial Sequence
  synthetic polypeptide
GGSGQRGRKALE

SEQ ID NO: 43 - protein, 12 aa, Artificial Sequence
  synthetic polypeptide
SLSALLSSDIFN

SEQ ID NO: 44 - protein, 12 aa, Artificial Sequence
  synthetic polypeptide
SLPRFKIIGGFN

SEQ ID NO: 45 - protein, 12 aa, Artificial Sequence
  synthetic polypeptide
SLLGIAVPGNFN

SEQ ID NO: 46 - protein, 12 aa, Artificial Sequence
  synthetic polypeptide
FFKNIVTPRTPP

SEQ ID NO: 47 - protein, 8 aa, Artificial Sequence
  synthetic polypeptide
GPLGMHGK

SEQ ID NO: 48 - protein, 9 aa, Artificial Sequence
  synthetic polypeptide
GPLGLSLGK